(19) United States
(12) Patent Application Publication
(10) Pub. No.: US 2014/0342419 A1
(43) Pub. Date: Nov. 20, 2014

(54) MICROORGANISM STRAINS FOR THE PRODUCTION OF 2,3-BUTANEDIOL

(71) Applicant: Metabolic Explorer, Saint Beauzire (FR)

(72) Inventors: Wanda Dischert, Vic-Le-Comte (FR); Guillaume Letellier, Montferrier-sur-Lez (FR); Rainer Figge, Le Crest (FR); Véronique Douchin, Copenhagen (FR)

(21) Appl. No.: 14/359,202

(22) PCT Filed: Nov. 21, 2012
(86) PCT No.: PCT/EP2012/073242

§ 371 Date: May 19, 2014

Related U.S. Application Data
(60) Provisional application No. 61/562,136, filed on Nov. 21, 2011.

(30) Foreign Application Priority Data
Nov. 21, 2011 (EP) 11190018.9

(51) Int. Cl. C12P 7/18; C12N 9/04

(52) U.S. Cl. CPC C12P 7/18; C12N 9/0006; C12Y 101/01001
USPC 435/158; 435/254.21; 435/252.3

(57) ABSTRACT

The present invention is related to a recombinant microorganism engineered for the production of 2,3-butanediol (BDO), wherein said microorganism overexpresses at least one gene encoding a polypeptide involved in the conversion of pyruvate into 2,3-butanediol. The invention is also related to a method of production of 2,3-butanediol comprising the following steps:
providing a recombinant microorganism as described above,
cultivating the recombinant microorganism in a culture medium containing a source of carbon, and
recovering the 2,3-butanediol.

### TECHNICAL FIELD OF THE INVENTION

The present invention provides non-naturally occurring microorganism having a 2,3-butanediol pathway. The recombinant microorganism is modified to improve the production of 2,3-butanediol compared to the unmodified microorganism. The invention also provides methods for using such microorganisms to produce 2,3-butanediol.

### BACKGROUND OF THE INVENTION

As crude oil reserves become increasingly scarce, bio-refinery systems that integrate biomass conversion processes and equipment to produce fuels, power, and chemicals from annually renewable resources are at the stage of worldwide development. Many chemicals that could only be produced by traditional chemical processes in the past can now have the potential to be generated biologically, using renewable resources (Danner & Braun, 1999; Hatti-Kaul et al., 2007). Microbial production of 2,3-butanediol (2,3-BDO) is one such example. Interest in this bioprocess has increased remarkably because 2,3-BDO has a large number of industrial applications, and microbial production will alleviate the dependence on oil supply for the production of platform chemicals (Celińska & Grajek, 2009; Wu et al., 2008).

2,3-BDO is also known as 2,3-butylene glycol, dimethylene glycol, dimethylethylene glycol and the IUPAC name is butane-2,3-diol. Its molecular formula is C₄H₁₀O₂. The CAS number is 513-85-9.

2,3-BDO exists in 3 isomeric forms: D-(−)-, L-(+)- and meso- as shown below:

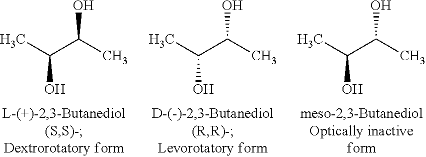

One possible application of 2,3-BDO is its conversion into 1,3-butadiene, that can be used in synthetic rubber production. Additionally, 2,3-BDO has potential applications in the manufacture of printing inks, perfumes, fumigants, moistening and softening agents, explosives, plasticizers, food, and pharmaceuticals (Garg & Jain, 1995; Syu, 2001).

Microbial 2,3-BDO production has a history of more than 100 years. It was first investigated in 1906 by Harden and Walpole and in 1912 by Harden and Norris (Magee & Kosaric, 1985). The bacterium employed in these early studies was Klebsiella pneumoniae (formerly Aerobacter aerogenes or Klebsiella aerogenes). Since then, the production of 2,3-BDO has been progressing and is well described in bacteria (Celińska & Grajek, 2009; Ji et al., 2011), mainly risk group 2 and risk group 1 bacteria, including E. coli (Ui et al., 2004; Nielsen et al., 2010).

2,3-butanediol is a by-product of alcoholic fermentation by yeast and usually one of the most abundant minor constituent of wine. It originates from the reduction of acetoin (Romano & Suzzi, 1996). Numerous attempts have been made to engineer Saccharomyces cerevisiae strains with reduced acetoin yields, by re-orienting carbons toward glycerol and 2,3-BDO to obtain low-alcohol yeasts with desirable organoleptic features, permitting the decrease of the ethanol contents in wines by up to 3° C. (Ehsani et al., 2009; Gonzalez et al., 2010).

Yeast cells have been more recently considered as a cellular mini bioreactor to produce 2,3-BDO. For instance, the patent application WO2011041426 provides yeast cells that are engineered to have improved growth and production of products, among them the 2,3-BDO. Particularly, in these yeast cells one or more pyruvate decarboxylase genes have been inactivated in order to suppress the endogenous competing pyruvate-utilizing metabolic pathway. Another examples of 2,3-BDO production are described in patent application WO2010151525 and in the work of Ng et al. (2012).

Generally, yeast can grow rapidly and can be cultivated at higher density as compared with bacteria, and does not require an aseptic environment in the industrial setting. Furthermore, yeast cells can be more easily separated from the culture medium compared to bacterial cells, greatly simplifying the process for product extraction and purification.

The present inventors propose alternative pathways in order to improve 2,3-butanediol biosynthesis in microorganisms, including yeasts and bacteria, regarding environmental impact, performance of production, facility of recovery or economy.

### SUMMARY OF THE INVENTION

The present invention is related to new alternative pathways for the fermentative production of 2,3-butanediol and provides non-naturally occurring microorganism having a 2,3-butanediol pathway.

In particular, the invention is related to a recombinant microorganism engineered to produce 2,3-butanediol (BDO) wherein said microorganism is chosen among bacterium or yeast and overexpresses at least one gene encoding a polypeptide involved in the conversion of pyruvate into 2,3-butanediol. According to the invention, 2,3-butanediol can be obtained via six different metabolic pathways.

The first pathway of the invention comprises genes encoding polypeptides having activity of acetolactate synthase (ALS), acetolactate decarboxylase (ALDC) and butanediol dehydrogenase (BDH).

The second pathway of the invention involves genes encoding a polypeptide catalysing the conversion of pyruvate to acetaldehyde, polypeptides catalysing the conversion of pyruvate to acetyl-CoA and the conversion of acetyl-CoA to acetaldehyde, a polypeptide catalysing the condensation of pyruvate and/or acetaldehyde to acetoin and a polypeptide having butanediol dehydrogenase (BDH) activity, thus reducing acetoin to 2,3-butanediol.

The third pathway of the invention comprises genes encoding polypeptides having activity of acetolactate synthase (ALS) and butanediol dehydrogenase (BDH).

The fourth pathway involves genes encoding polypeptides having activity of pyruvate decarboxylase (PDC) and pyruvate formate lyase (PFL), acetoin dehydrogenase (ACDH) and butanediol dehydrogenase (BDH).

The fifth pathway of the invention comprises genes encoding polypeptides having activity of propionate CoA transferase, beta-keto-thiolase, thioesterase, beta-keto-acid decarboxylase and butanediol dehydrogenase (BDH).

The sixth pathway of the invention involves genes encoding polypeptides having activity of acetolactate synthase (ALS), 3-hydroxy alkanoate decarboxylase, dehydratase, acetolactate decarboxylase and butanediol dehydrogenase (BDH).

In another aspect, the invention relates also to a method of production of 2,3-butanediol which comprises providing a recombinant microorganism, cultivating the recombinant microorganism in a culture medium containing a source of carbon, and recovering the 2,3-butanediol.

### BRIEF DESCRIPTION OF THE DRAWINGS

FIG. 1 shows the first metabolic pathway for 2,3-butanediol named Pathway 1

FIG. 2 shows the second metabolic pathway for 2,3-butanediol named Pathway 2

FIG. 3 shows the third metabolic pathway for 2,3-butanediol named Pathway 3

FIG. 4 shows the fourth metabolic pathway for 2,3-butanediol named Pathway 4

FIG. 5 shows the fifth metabolic pathway for 2,3-butanediol named Pathway 5

FIG. 6 shows the sixth metabolic pathway for 2,3-butanediol named Pathway 6

### DETAILED DESCRIPTION OF THE INVENTION

Before describing the present invention in detail, it is to be understood that this invention is not limited to particularly exemplified methods and may, of course, vary. It is also to be understood that the terminology used herein is for the purpose of describing particular embodiments of the invention only, and is not intended to be limiting, which will be limited only by the appended claims.

All publications, patents and patent applications cited herein, whether supra or infra, are hereby incorporated by reference in their entirety. However, publications mentioned herein are cited for the purpose of describing and disclosing the protocols, reagents and vectors that are reported in the publications and that might be used in connection with the invention.

Furthermore, the practice of the present invention employs, unless otherwise indicated, conventional microbiological and molecular biological techniques within the skill of the art. Such techniques are well known to the skilled worker, and are explained fully in the literature. See, for example, Guthrie & Fink, 2004 and Sambrook et al., (1999) (2001).

It must be noted that as used herein and in the appended claims, the singular forms “a”, “an”, and “the” include plural reference unless the context clearly dictates otherwise. Thus, for example, a reference to “a microorganism” includes a plurality of such microorganisms, and a reference to “an endogenous gene” is a reference to one or more endogenous genes, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

In the claims that follow and in the preceding description of the invention, except where the context requires otherwise due to express language or necessary implication, the terms “comprise”, “have”, “contain” or “include” or variations such as “comprises”, “comprising”, “includes”, “including”, “has”, “having”, “contains” or “containing” are used in an inclusive sense, i.e. to specify the presence of the stated features but not to preclude the presence or addition of further features in various embodiments of the invention.

### DEFINITIONS

The terms 2,3-butanediol, 2,3-BDO or BDO are used interchangeably in the invention and refer to butane-2,3-diol, also called dimethylene glycol.

The term “microorganism”, as used herein, refers to a bacterium, yeast or fungus which is not modified artificially. The microorganism may be “donor” if it provides genetic element to be integrated in the microorganism “acceptor” which will express this foreign genetic element or if it used as tool for genetic constructions or protein expressions.

The microorganism of the invention is chosen among bacterium, yeast or fungus which expresses genes for the biosynthesis of 2,3-butanediol.

Preferentially, the bacterium of the invention is selected among Enterobacteriaceae, Bacillaceae, Streptomycetaceae and Corynebacteriaceae. More preferentially the microorganism is a species of Escherichia, Pantoea, Salmonella, or Corynebacterium. Even more preferentially the microorganism is either the species Escherichia coli or Corynebacterium glutamicum.

Preferentially, the yeast of the invention is selected among the genera Saccharomycetaceae, Candida, Kluyveromyces, Ashbya, Dekkera, Pichia (Hansenula), Debaryomyces, Clavispora, Lodderomyces, Yarrowia, Schizosaccharomyces, Cryptococcus, Malassezia. More preferentially the yeast is a species of Saccharomyces, Pichia, Kluyveromyces or Yarrowia. According to a specific aspect of the invention, the yeast is from the species Saccharomyces cerevisiae, Kluyveromyces lactis or Kluyveromyces marxianus.

More preferentially, the bacterium is Escherichia coli and the yeast is Saccharomyces cerevisiae.

The term “recombinant microorganism” or “genetically modified microorganism” or “recombinant yeast” or “genetically modified yeast” or “recombinant bacterium” or “genetically modified bacterium”, as used herein, refers to a microorganism, including yeast and bacteria, genetically modified or genetically engineered. It means, according to the usual meaning of these terms, that the microorganism of the invention is not found in nature and is modified either by introduction or by deletion or by modification of genetic elements from equivalent microorganism found in nature. It can also be transformed by forcing the development and evolution of new metabolic pathways by combining directed mutagenesis and evolution under specific selection pressure (see for instance WO2004076659).

A microorganism may be modified to express exogenous genes if these genes are introduced into the microorganism with all the elements allowing their expression in the host microorganism. A microorganism may be modified to modulate the expression level of an endogenous gene. The modification or “transformation” of microorganism like bacterium or yeast with exogenous DNA is a routine task for those skilled in the art.

The term “endogenous gene” means that the gene was present in the microorganism before any genetic modification, in the wild-type strain. Endogenous genes may be overexpressed by introducing heterologous sequences in addition to, or to replace endogenous regulatory elements, or by introducing one or more supplementary copies of the gene into the chromosome or a plasmid. Endogenous genes may also be modified to modulate their expression and/or activity. For example, mutations may be introduced into the coding sequence to modify the gene product or heterologous sequences may be introduced in addition to or to replace endogenous regulatory elements. Modulation of an endogenous gene may result in the up-regulation and/or enhancement of the activity of the gene product, or alternatively, in the down-regulation and/or attenuation of the activity of the endogenous gene product. Another way to enhance expression of endogenous genes is to introduce one or more supplementary copies of the gene onto the chromosome or a plasmid.

The term “exogenous gene” means that the gene was introduced into a microorganism, by means well known by the man skilled in the art, whereas this gene is not naturally occurring in the microorganism. Exogenous genes can be heterologous or not. Microorganism can express exogenous genes if these genes are introduced into the microorganism with all the elements allowing their expression in the host microorganism. Transforming microorganisms with exogenous DNA is a routine task for the man skilled in the art. Exogenous genes may be integrated into the host chromosome, or be expressed extra-chromosomally from plasmids or vectors. A variety of plasmids, which differ with respect to their origin of replication and their copy number in the cell, are all known in the art. These genes may be heterologous or homologous. The sequence of exogenous genes may be adapted for its expression in the host microorganism. Indeed, the man skilled in the art knows the notion of codon usage bias and how to adapt nucleic sequences for a particular codon usage bias without modifying the deduced protein.

The term “heterologous gene” means that the gene is derived from a species of microorganism different from the recipient microorganism that expresses it. It refers to a gene which is not naturally occurring in the microorganism.

In the present application, all genes are referenced with their common names and with references that give access to their nucleotidic sequences on the websites http://www.ebi.ac.uk/embl/ or http://www.ncbi.nlm.nih.gov/gene. Using the references given in Genbank for known genes, those skilled in the art are able to determine the equivalent genes in other organisms, bacterial strains, yeast, fungi, mammals, plants, etc. This routine work is advantageously done using consensus sequences that can be determined by carrying out sequence alignments with genes derived from other microorganisms and designing degenerated probes to clone the corresponding gene in another organism.

The man skilled in the art knows different means to modulate, and in particular up-regulate, the expression of endogenous genes. For example, a way to enhance expression of endogenous genes is to introduce one or more supplementary copies of the gene onto the chromosome or a plasmid.

Another way is to replace the endogenous promoter of a gene with a stronger promoter. These promoters may be homologous or heterologous.

Homologous promoters known to allow a high level of expression in yeast are the ones expressing glycolytic genes; ADH1, GPDH, TEF1, truncated HXT7, PFK1, FBA1, PGK1 and TDH3 etc. . . . More preferably, the truncated HXT7 promoter is used in the yeasts of the invention. In yeast, nucleic acid expression construct preferably comprises regulatory sequences, such as promoter and terminator sequences, which are operatively linked with the nucleic acid sequence coding for the plant pentose transporter.

Preferred promoter sequences are HXT7, truncated HXT7, PFK1, FBA1, PGK1, ADH1 and TDH3. Preferred terminator sequences are CYC1, FBA1, PGK1, PFK1, ADH1 and TDH3. The nucleic acid expression construct may further comprise 5′ and/or 3′ recognition sequences and/or selection markers.

It is well within the ability of the person skilled in the art to select appropriate promoters, for example, the promoters from the collection comprising 11 mutants of the strong constitutive Saccharomyces cerevisiae TEF1 promoter are widely used (Nevoigt et al., 2007).

Homologous promoters known to allow a high level of expression in bacterium are, for instance promoters Ptrc, Ptac, Plac, or the lambda promoter cI which are widely used. These promoters can be “inducible” by a particular compound or by specific external condition like temperature or light. These promoters may be homologous or heterologous.

The term ‘overexpression’ means in this context that the expression of a gene or an enzyme is increased compared to the non-modified microorganism. Increasing the expression of an enzyme is obtained by increasing the expression of a gene encoding said enzyme.

The ‘activity’ of an enzyme is used interchangeably with the term ‘function’ and designates, in the context of the invention, the reaction that is catalyzed by the enzyme.

The terms “reduced activity” or “attenuated activity” of an enzyme mean either a reduced specific catalytic activity of the protein obtained by mutation in the aminoacids sequence and/or decreased concentrations of the protein in the cell obtained by mutation of the nucleotidic sequence or by deletion of the coding gene.

The term ‘enhanced activity’ of an enzyme designates either an increased specific catalytic activity of the enzyme, and/or an increased quantity/availability of the enzyme in the cell, obtained for example by overexpression of the gene encoding the enzyme.

The terms “encoding” or “coding” refer to the process by which a polynucleotide, through the mechanisms of transcription and translation, produces an amino-acid sequence.

The gene(s) encoding the enzyme(s) can be exogenous or endogenous.

“Attenuation” of genes means that genes are expressed at an inferior rate than the non-modified rate. The attenuation may be achieved by means and methods known to the man skilled in the art and contains gene deletion obtained by homologous recombination, gene attenuation by insertion of an external element into the gene or gene expression under a weak promoter. The man skilled in the art knows a variety of promoters which exhibit different strengths and which promoter to use for a weak genetic expression.

The methods of the present invention require the use of one or more chromosomal integration constructs for the stable introduction of a heterologous nucleotide sequence into a specific location on a chromosome or for the functional disruption of one or more target genes in a genetically modified microbial cell. In some embodiments, disruption of the target gene prevents the expression of a functional protein. In some embodiments, disruption of the target gene results in the expression of a non-functional protein from the disrupted gene.

Parameters of chromosomal integration constructs that may be varied in the practice of the present invention include, but are not limited to, the lengths of the homologous sequences; the nucleotide sequence of the homologous sequences; the length of the integrating sequence; the nucleotide sequence of the integrating sequence; and the nucleotide sequence of the target locus. In some embodiments, an effective range for the length of each homologous sequence is 20 to 5,000 base pairs, preferentially 40 base pairs. In particular embodiments, the length of each homologous sequence is about 500 base pairs. For more information on the length of homology required for gene targeting, see Hasty et al., (1991). In some embodiments, the expression vector or chromosomal integration vector used to genetically modify a microbial cell of the invention comprises one or more selectable markers useful for the selection of transformed microbial cells.

In some embodiments, the selectable marker is an antibiotic resistance marker. Illustrative examples of antibiotic resistance markers include, but are not limited to the BLA, NAT1, PAT, AUR1-C, PDR4, SMR1, CAT, mouse dhfr, HPH, DSDA, KAN^{R}, and SH BLE gene products. The BLA gene product from E. coli confers resistance to beta-lactam antibiotics and to all the anti-gram-negative-bacterial penicillins except temocillin; the NAT1 gene product from S. noursei confers resistance to nourseothricin; the PAT gene product from S. viridochromogenes Tu94 confers resistance to bialophos; the AUR1-C gene product from Saccharomyces cerevisiae confers resistance to Auerobasidin A (AbA); the PDR4 gene product confers resistance to cerulenin; the SMR1 gene product confers resistance to sulfometuron methyl; the CAT gene product from the Tn9 transposon confers resistance to chloramphenicol; the mouse dhfr gene product confers resistance to methotrexate; the HPH gene product of Klebsiella pneumonia confers resistance to Hygromycin B; the DSDA gene product of E. coli allows cells to grow on plates with D-serine as the sole nitrogen source; the KAN^{R} gene of the Tn903 transposon confers resistance to G418; and the SH BLE gene product from Streptoalloteichus hindustanus confers resistance to Zeocin (bleomycin). In some embodiments, the antibiotic resistance marker is deleted after the genetically modified microbial cell of the invention is isolated. The man skilled in the art is able to choose suitable marker in specific genetic context.

In some embodiments, the selectable marker rescues an auxotrophy (e.g., a nutritional auxotrophy) in the genetically modified microbial cell. In such embodiments, a parent microbial cell comprises a functional disruption in one or more gene products that function in an amino acid or nucleotide biosynthetic pathway, such as, for example, the HIS3, LEU2, LYS1, LYS2, MET15, TRP1, ADE2, and URA3 gene products in yeast, which renders the parent microbial cell incapable of growing in media without supplementation with one or more nutrients (auxotrophic phenotype). The auxotrophic phenotype can then be rescued by transforming the parent microbial cell with an expression vector or chromosomal integration encoding a functional copy of the disrupted gene product, and the genetically modified microbial cell generated can be selected for based on the loss of the auxotrophic phenotype of the parent microbial cell. Utilization of the URA3, TRP1, and HIS3 genes as selectable markers has a marked advantage because both positive and negative selections are possible. Positive selection is carried out by auxotrophic complementation of the URA3, TRP1, and LYS2 mutations, whereas negative selection is based on specific inhibitors, i.e., 5-fluoro-orotic acid (FOA), 5-fluoroanthranilic acid, and a-aminoadipic acid (aAA), respectively, that prevent growth of the prototrophic strains but allows growth of the URA3, TRP1, and HIS3 mutants, respectively. Preferentially, the selectable marker gene used is HIS3.

Suitable promoters, transcriptional terminators, and coding regions may be cloned into E. coli-yeast shuttle vectors, and transformed into yeast cells as described in Examples. These vectors allow propagation in both E. coli and yeast strains. The vector contains a selectable marker and sequences allowing autonomous replication, or chromosomal integration in the desired host. Used plasmids in yeast are for example shuttle vectors p423H7, p424H7, p425H7 et p426H7 (Wieczorke et al., 1999 and Hamacher et al., 2002) which contain a E. coli replication origin, a yeast origin of replication, and a marker for nutritional selection. The selection markers for these four vectors are HIS3, TRP1, LEU2 and URA3.

In other embodiments, the selectable marker rescues other non-lethal deficiencies or phenotypes that can be identified by a known selection method.

The “fermentation” or “culture” is generally conducted in fermenters with an appropriate culture medium adapted to the microorganism being cultivated, containing at least one simple carbon source, and if necessary co-substrates.

Microorganisms disclosed herein may be grown in fermentation media for the production of a product from pyruvate. For maximal production of some products such as 2,3-butanediol, the microorganism strains used as production hosts preferably have enhanced tolerance to the produced chemical, and have a high rate of carbohydrate utilization. These characteristics may be conferred by mutagenesis and selection, genetic engineering, or may be natural.

Fermentation media, or ‘culture medium’, for the present cells contain at least about 2 g/L glucose. Additional carbon substrates may include but are not limited to monosaccharides such as fructose, mannose, xylose and arabinose, oligosaccharides such as lactose maltose, galactose, or sucrose, polysaccharides such as starch or cellulose or mixtures thereof and unpurified mixtures from renewable feedstocks such as cheese whey permeate cornsteep liquor, sugar beet molasses, and barley malt. Other carbon substrates may include ethanol, lactate, succinate, or glycerol. Hence it is contemplated that the source of carbon utilized in the present invention may encompass a wide variety of carbon containing substrates and will only be limited by the choice of organism. In a specific embodiment of the invention, the substrate may be hydrolysate of second generation carbons sources such wheat straw, miscanthus plants, sugar cane bagasses, wood and others that are known to the man skilled in the art.

Although it is contemplated that all of the above mentioned carbon substrates and mixtures thereof are suitable in the present invention, preferred carbon substrates are glucose, fructose, and sucrose, or mixtures of these with C5 sugars such as xylose and/or arabinose for microorganisms modified to use C5 sugars.

In addition to an appropriate carbon source, fermentation media must contain suitable minerals, salts, cofactors, buffers and other components, known to those skilled in the art, suitable for the growth of the cultures and promotion of the enzymatic pathway necessary for the production of the desired product.

The terms “anaerobic conditions” refer to conditions under which the oxygen concentration in the culture medium is too low for the microorganism to be used as terminal electron acceptor. Anaerobic conditions may be achieved either by sparging the culture medium with an inert gas such as nitrogen until oxygen is no longer available to the microorganism or by the consumption of the available oxygen by the microorganism.

“Aerobic conditions” refers to concentrations of oxygen in the culture medium that are sufficient for an aerobic or facultative anaerobic microorganism to use as a terminal electron acceptor.

“Microaerobic condition” refers to a culture medium in which the concentration of oxygen is less than that in air, i.e; oxygen concentration up to 6% O₂.

An “appropriate culture medium” designates a medium (e.g., a sterile, liquid media) comprising nutrients essential or beneficial to the maintenance and/or growth of the cell such as carbon sources or carbon substrate, nitrogen sources, for example, peptone, yeast extracts, meat extracts, malt extracts, urea, ammonium sulfate, ammonium chloride, ammonium nitrate and ammonium phosphate; phosphorus sources, for example, monopotassium phosphate or dipotassium phosphate; trace elements (e.g., metal salts), for example magnesium salts, cobalt salts and/or manganese salts; as well as growth factors such as amino acids, vitamins, growth promoters, and the like.

The term “carbon source” or “carbon substrate” or “source of carbon” according to the present invention denotes any source of carbon that can be used by those skilled in the art to support the normal growth of a microorganism, including hexoses (such as glucose, galactose or lactose), pentoses, monosaccharides, oligosaccharides, disaccharides (such as sucrose, cellobiose or maltose), molasses, starch or its derivatives, cellulose, hemicelluloses and combinations thereof.

### 2,3-Butanediol Biosynthesis Pathways

The present invention is related to a recombinant microorganism engineered to express a 2,3-butanediol pathway, by overexpressing at least one gene encoding a polypeptide involved in the conversion of pyruvate to 2,3-butanediol.

Preferentially, the recombinant microorganism of the invention overexpresses at least one exogenous gene involved in the 2,3-butanediol biosynthesis.

In one embodiment of the invention, the recombinant microorganism exhibits a reduced production of 2,3-butanediol by-products.

In yeast, the main 2,3-butanediol by-product is ethanol. Thus, activity of at least one enzyme catalysing the conversion of acetaldehyde to ethanol chosen among ADH1, ADH2, ADH3, ADH4, ADH5, ADH6, ADH7 and SFA1 is reduced.

Yeast is known as a very efficient ethanol producer during fermentation. Ethanol is produced from pyruvate by an enzyme having alcohol dehydrogenase (ADH) activity. Eight different ADH are present in yeast: ADH1, ADH2, ADH3, ADH4, ADH5, ADH6, ADH7 and SFA1 encoded respectively by ADH1, ADH2, ADH3, ADH4, ADH5, ADH6, ADH7 and SFA1 genes (Smidt et al., 2012). To increase the productivity of 2,3-butanediol, it is necessary to decrease the ethanol production.

Preferentially, the recombinant yeast cell exhibits a reduced activity of at least one enzyme catalysing the conversion of acetaldehyde to ethanol, chosen among: ADH1, ADH2, ADH3, ADH4, ADH5, ADH6, ADH7 and SFA1.

In particular, at least the activity of ADH1 is reduced. In a preferred embodiment of the invention the activities of ADH1, ADH2, ADH3, ADH4, ADH5 and SFA1 are reduced in the recombinant yeast cell. More preferably, the activities of ADH1, ADH3, ADH5 are reduced in the recombinant yeast cell. Preferably, ADH activities are reduced by attenuating expression of the corresponding coding genes. In particular, according to the invention the ADH1 gene is deleted in the recombinant yeast cell. More preferably, the ADH1, ADH2, ADH3, ADH4, ADH5 and SFA1 genes are deleted. Even more preferably, the ADH1, ADH3 and ADH5 genes are deleted.

In bacterium, the main 2,3-butanediol by-products are lactate and acetate. Thus, activity of at least one enzyme catalysing the conversion of pyruvate to lactate or acetate is reduced. Preferably, activity of at least one enzyme chosen among LdhA, PoxB, AckA, Pta or AceEF is reduced. More preferably, activities of all LdhA, PoxB, AckA and Pta are reduced.

Preferably, such activities are reduced by attenuating expression of the corresponding coding genes. In particular, expression of at least one gene chosen among ldhA, poxB, ackA, pta or aceEF is attenuated. More particularly, all genes ldhA, poxB, ackA and pta are deleted.

According to the invention, the 2,3-butanediol is produced in a recombinant microorganism via six different metabolic pathways, that are described below.

Pathway 1

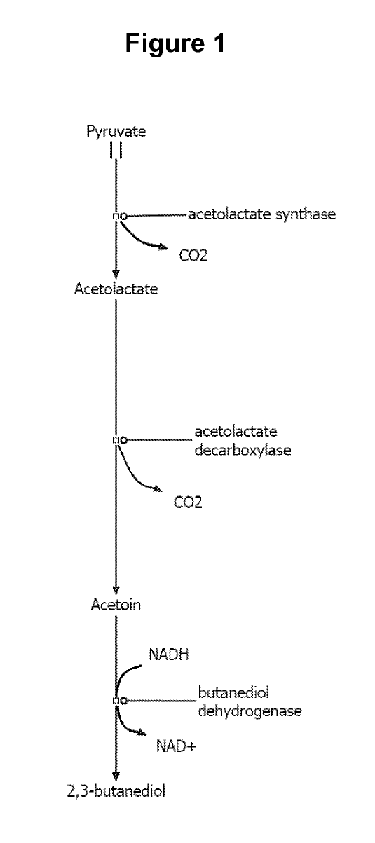

The first pathway of 2,3-butanediol biosynthesis (FIG. 1) includes the following successive steps of: a) conversion of pyruvate to acetolactate by acetolactate synthase (ALS), b) conversion of acetolactate to acetoin by acetolactate decarboxylase (ALDC) and c) conversion of acetoin to 2,3-butanediol by butanediol dehydrogenase (BDH).

In an aspect of the invention, the recombinant microorganism expresses genes encoding polypeptides having an activity of acetolactate synthase (ALS), acetolactate decarboxylase (ALDC) and butanediol dehydrogenase (BDH).

The different genes are endogenous genes or exogenous genes. The man skilled in the art knows that polypeptide having ALS, ALDC or BDH activity isolated from a variety of organisms may be used in the present recombinant microorganism. Identification of similar nucleotides or amino acids sequences using bioinformatics algorithms such as BLAST (Altschul et al., 1993) on publicly available databases with known sequences is well done by the skilled person.

ALS enzymes which may also be called acetohydroxy acid synthases and belong to the class Enzyme Class 2.2.1.6 are well known by the man skilled in the art, as they participate in the biosynthetic pathway for the proteinogenic amino acids leucine and valine and in the fermentative production of 2,3-butanediol in many organisms.

In the present invention, ALS enzymes that have substrate specificity for pyruvate over ketopyruvate are preferred.

Enzymes having ALS activity are encoded by one of the following genes: ilvB and ilvN from Corynebacterium glutamicum, ilvB and ilvN from E. coli, budB and alsS from Klebsiella pneumoniae 342, budB from Klebsiella oxytoca, budB from Enterobacter aerogenes, slaB from Serratia marcescens, ILV2 and ILV6 from Saccharomyces cerevisiae, alsS from Bacillus subtilis subtilis 168, alsS from Bacillus thuriengensis or PPSC2_c1472 from Bacillus polymyxa. Any ALS coding gene having at least 80-85%, preferentially 85-90%, more preferentially 90-95%, even more preferentially 96%, 97%, 98%, 99% sequence identity to any of those genes may be used.

In a preferred embodiment of the invention the expressed ALS enzyme is encoded by the gene budB from Klebsiella pneumoniae.

In another preferred embodiment the expressed ALS enzyme is encoded by the genes ILV2 or ILV6 from Saccharomyces cerevisiae.

More preferentially, in the bacterium of the invention ALS enzyme is encoded by the gene budB from Klebsiella pneumoniae whereas in the yeast of the invention ALS enzyme is encoded by the gene ILV2 from Saccharomyces cerevisiae.

In order to optimize 2,3-butanediol biosynthesis in yeast the ALS is specifically expressed in the cytosol. This cytosolic expression is achieved by transforming the yeast with a gene encoding an ALS lacking the mitochondrial targeting signal sequence as described in Brat et al. (2012).

ALDC enzymes belonging to Enzyme Class 4.1.1.5 and being naturally absent from yeast are encoded by the genes budA from Klebsiella pneumoniae, budA from Klebsiella oxytoca, budA from Enterobacter aerogenes, salA from Serratia marcescens, alsD from Bacillus subtilis subtilis 168, alsD from Bacillus thuriengiensis or PPSC2_c2281 from Bacillus polymyxa. Any ALDC encoding gene having at least 80-85%, preferentially 85-90%, more preferentially 90-95%, even more preferentially 96%, 97%, 98%, 99% sequence identity to any of those genes may be used.

In a preferred embodiment of the invention the expressed ALDC enzyme is encoded by the gene budA from Klebsiella pneumoniae in the bacterium and the yeast of this invention.

BDH enzymes may also be called acetoin reductase. BDH enzymes may have specificity for the production of (R)-2,3-butanediol (EC 1.1.1.4) or (S)-2,3-butanediol (EC 1.1.1.76). If one of the two forms is preferred, the man skilled in the art will know which enzyme to choose and to overproduce in the recombinant cell to promote the specific production.

Enzymes having BDH activity are in particular encoded by one of the following genes: butA from Corynebacterium glutamicum, budC from Klebsiella pneumoniae, budC from Klebsiella oxytoca, EAE_{—}17490 from Enterobacter aerogenes, slaC from Serratia marcescens, BDH1 and BDH2 from Saccharomyces cerevisiae, bdhA from Bacillus subtilis subtilis 168, BMB171_C0587 from Bacillus thuriengensis or PPSC2_c3335 and PPSC2_c3890 from Bacillus polymyxa. Any BDH coding gene having at least 80-85%, preferentially 85-90%, more preferentially 90-95%, even more preferentially 96%, 97%, 98%, 99% sequence identity to any of those genes may be used.

In a preferred embodiment of the invention the expressed BDH enzyme is encoded by the gene budC from Klebsiella pneumoniae.

In another preferred embodiment the expressed BDH enzyme is encoded by the genes BDH1 and BDH2 from Saccharomyces cerevisiae.

More preferentially, in the bacterium of the invention BDH enzyme is encoded by the gene budC from Klebsiella pneumoniae whereas in the yeast of the invention BDH enzyme is encoded by the gene BDH1 from Saccharomyces cerevisiae or BDH2 from Saccharomyces cerevisiae.

In a preferred embodiment of the invention, the recombinant bacterium of the invention expresses genes budB, budA and budC from Klebsiella pneumoniae, whereas endogenous genes encoding ALS, ALDC and BDH are deleted.

In another preferred embodiment, the recombinant yeast of the invention expresses the budA gene from Klebsiella pneumoniae, ILV2 and BDH1 genes from Saccharomyces cerevisiae. Preferably, the recombinant yeast cell exhibits reduced activity of at least one enzyme catalysing the conversion of pyruvate to acetaldehyde in way to improve the conversion of pyruvate to acetolactate. Preferentially the expression of at least one gene chosen among PDC1, PDC5 or PDC6 is attenuated in the yeast of the invention. More preferentially the PDC1 gene is deleted. More preferentially the genes PDC1, PDC5 and PDC6 are deleted in the modified yeast.

Pathway 2

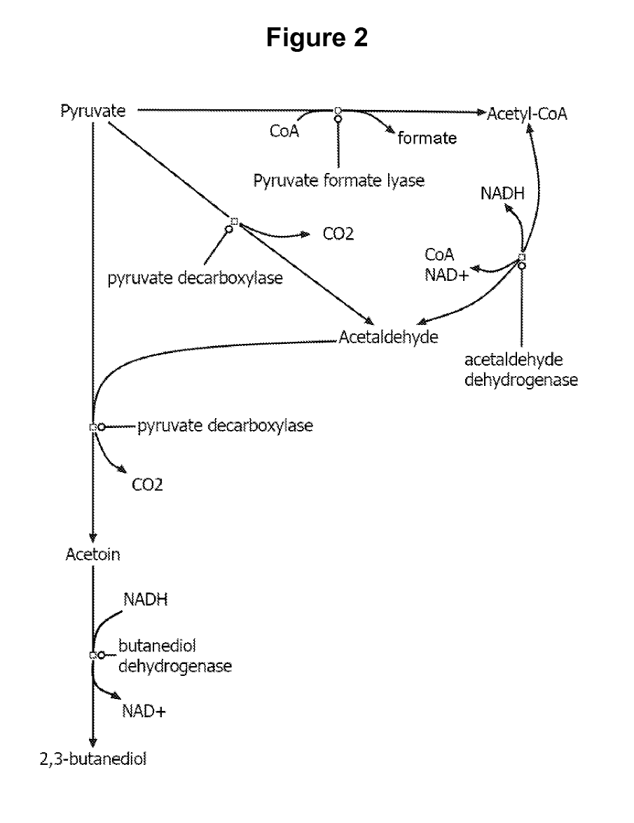

The second pathway of 2,3-butanediol biosynthesis (FIG. 2) includes the successive steps of conversion of pyruvate to acetaldehyde and/or to acetyl-CoA, conversion of acetyl-CoA to acetaldehyde, conversion of pyruvate and/or acetaldehyde to acetoin and conversion of acetoin to 2,3-butanediol by butanediol dehydrogenase (BDH).

In this invention, the terms “conversion of pyruvate and/or acetaldehyde to acetoin” refers either to the conversion of one molecule of pyruvate and one molecule of acetaldehyde into acetoin, or to the condensation of two molecules of acetaldehyde into acetoin.

The recombinant microorganism expresses the following enzymes:
a) A polypeptide catalysing the conversion of pyruvate to acetaldehyde or two polypeptides, one catalysing the conversion of pyruvate to acetyl-CoA and the other catalysing the conversion of acetyl-CoA to acetaldehyde, and
b) a polypeptide catalysing the condensation of pyruvate and acetaldehyde to acetoin or a polypeptide catalysing the condensation of acetaldehyde to acetoin, and
c) a butanediol dehydrogenase.

In one aspect, the recombinant microorganism of the invention expresses genes encoding polypeptides having activity of conversion of pyruvate to acetaldehyde, conversion of pyruvate and/or acetaldehyde to acetoin and butanediol dehydrogenase (BDH).

The conversion of pyruvate to acetaldehyde and of pyruvate and/or acetaldehyde to acetoin may be achieved by the same enzyme: the pyruvate decarboxylase (PDC). PDC enzyme exhibits several activities: the major activity is the conversion of pyruvate to acetaldehyde and its secondary activity is the conversion of pyruvate and/or acetaldehyde to acetoin. The secondary activity of PDC allows the conversion of pyruvate and acetaldehyde to acetoin or the condensation of two acetaldehydes to acetoin (Romano & Suzzi, 1996). This second activity is named “acetoin synthase”.

In a preferred embodiment of the invention at least one of the PDC1, PDC5, PDC6 genes of Saccharomyces cerevisiae are overexpressed in the recombinant bacterium of the invention. More preferentially, PDC1 from Saccharomyces cerevisiae is overexpressed in the recombinant bacterium.

In another embodiment of the invention, the PDC enzyme may be evolved to enhance its acetoin synthase activity. The evolved enzymes are screened on their performance of conversion of pyruvate and/or acetaldehyde to acetoin. A preferred evolved PDC is a PDC which exhibit an improved activity of acetoin synthase of at least 20% compared to the non-evolved PDC.

In another aspect of the invention the PDC enzymes may be evolved to reduce its pyruvate decarboxylase activity, while keeping its acetoin synthase activity constant or increasing its acetoin synthase activity. In a preferred embodiment of the invention the ratio of acetoin synthase over pyruvate decarboxylase activity needs to be improved by at least a factor two, preferentially by a factor of ten and most preferred by a factor of 100, while increasing the acetoin synthase activity of the enzyme.

In another embodiment of the invention the acetoin synthase activity of PDC is replaced by an evolved glyoxylate carboxy-lyase enzyme (EC 4.4.1.47) or a novel enzyme exhibiting the same activity of conversion of pyruvate and/or acetaldehyde to acetoin. The evolved enzymes are obtained by evolution of an enzyme having carboxy-lyase activity and are screened on their specificity for their substrate. For example, the natural glyoxylate carboxy-lyase, encoded by the gcl gene from Escherichia coli, has substrate specificity for glyoxylate whereas the evolved glyoxylate carboxy-lyase presents substrate specificity for pyruvate and/or acetaldehyde.

In another embodiment of the invention, the acetaldehyde may be obtained by conversion of acetyl-CoA by acetaldehyde dehydrogenase. The acetaldehyde dehydrogenase is encoded for example by the mhpF gene from Escherichia coli K12, the dmpF gene from Pseudomonas sp or eutE from Salmonella enterica the acetaldehyde dehydrogenase activity residing in the N-terminus of E. coli adhE. Preferably the microorganism of the invention overexpresses mhpF gene from E. coli.

The used acetyl-CoA is obtained from pyruvate by pyruvate formate lyase activity which leads to the production of acetyl-CoA and formate. In this context, the recombinant microorganism will overexpress the pflB and/or pflA genes from Escherichia coli or any other pyruvate formate lyase enzyme known to the expert in the art.

In a further embodiment of the invention, formate may be recycling in the cell by overexpressing enzymatic complexes like FDH1 from Saccharomyces cerevisiae (Van Den Berg & Steensma 1997), FDH2 from Saccharomyces cerevisiae (Overkamp et al. 2002), fdh1 of Candida boidinii (Lu et al. 2009), formate hydrogen lyase complex (FHL) from Escherichia coli (Sawers 2005).

As above, enzymes having BDH activity are encoded by genes: butA from Corynebacterium glutamicum, budC from Klebsiella pneumoniae 342, budC from Klebsiella oxytoca, EAE_{—}17490 from Enterobacter aerogenes, slaC from Serratia marcescens, BDH1 and BDH2 from Saccharomyces cerevisiae, bdhA from Bacillus subtilis subtilis 168, BMB171_C0587 from Bacillus thuriengensis or PPSC2_c3335 and PPSC2_c3890 from Bacillus polymyxa. Any BDH coding gene having at least 80-85%, preferentially 85-90%, more preferentially 90-95%, even more preferentially 96%, 97%, 98%, 99% sequence identity to any of those genes may be used.

In a preferred embodiment of the invention the BDH used is encoded by the gene budC from Klebsiella pneumoniae.

In another preferred embodiment the expressed BDH enzyme is encoded by the genes BDH1 and/or BDH2 from Saccharomyces cerevisiae.

More preferentially, in the bacterium of the invention BDH enzyme is encoded by the gene budC from Klebsiella pneumoniae whereas in the yeast of the invention BDH enzyme is encoded by the gene BDH1 or BDH2 from Saccharomyces cerevisiae.

In wild type cells, in anaerobiosis, reductive power production by substrate catalysis is balanced with synthesis of reduced molecules, such as ethanol or acetate. In the present recombinant microorganisms, ethanol and acetate production pathways are disrupted. leading to an unbalanced redox state. Any method allowing an increase of NADH-dependent enzyme activity in the present production host cell may be used in balancing redox. For instance, addition of the pyruvate formate lyase PFLAB in absence of oxygen, allows the utilization of one more NADH during the conversion of acetyl-CoA into acetaldehyde and achieves the production of 2,3-BDO.

In a preferred embodiment, the recombinant bacterium of the invention overexpresses PDC1 gene from Saccharomyces cerevisiae, pflB and mhpF genes from Escherichia coli, and budC gene from Klebsiella pneumoniae, whereas endogenous genes encoding ALS, ALDC and BDH and poxB, ldhA, ackA and pta genes are deleted.

In another preferred embodiment, the recombinant yeast of the invention expresses the PDC1 gene from Saccharomyces cerevisiae and overexpressed pflA, pflB and mhpF genes from Escherichia coli, and BDH1 gene from Saccaromyces cerevisiae, whereas ADH1, ADH3, ADH5 and ILV2 endogenous genes from Saccharomyces cerevisiae are deleted.

Pathway 3

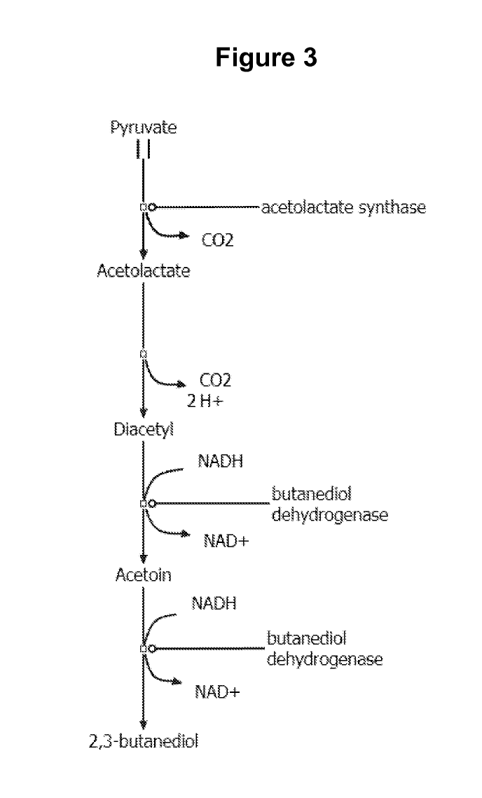

The third pathway of 2,3-butanediol biosynthesis (FIG. 3) includes steps of conversion of pyruvate to acetolactate by acetolactate synthase (ALS), conversion of acetolactate to diacetyl, conversion of diacetyl to acetoin and then acetoin to 2,3-butanediol by butanediol dehydrogenase (BDH).

In one embodiment, the recombinant microorganism of the invention expresses genes encoding polypeptides having activity of acetolactate synthase (ALS) and butanediol dehydrogenase (BDH).

As seen above, enzymes having ALS activity are encoded by genes: ilvB and ilvN from Corynebacterium glutamicum, ilvB and ilvN from Escherichia coli, budB and alsS from Klebsiella pneumoniae 342, budB from Klebsiella oxytoca, budB from Enterobacter aerogenes, slaB from Serratia marcescens, ILV2 and ILV6 from Saccharomyces cerevisiae, alsS from Bacillus subtilis subtilis 168, alsS from Bacillus thuriengensis or PPSC2_c1472 from Bacillus polymyxa. Any ALS coding gene having at least 80-85%, preferentially 85-90%, more preferentially 90-95%, even more preferentially 96%, 97%, 98%, 99% sequence identity to any of those genes may be used.

In a preferred embodiment of the invention the expressed ALS enzyme is encoded by the gene budB from Klebsiella pneumoniae.

In another preferred embodiment the expressed ALS enzyme is encoded by the genes ILV2 and ILV6 from Saccharomyces cerevisiae.

More preferentially, in the bacterium of the invention ALS enzyme is encoded by the gene budB from Klebsiella pneumoniae whereas in the yeast of the invention ALS enzyme is encoded by the gene ILV2 from Saccharomyces cerevisiae.

In order to optimize 2,3-butanediol biosynthesis in yeast the ALS is specifically expressed in the cytosol. This cytosolic expression is achieved by transforming the yeast with a gene encoding an ALS lacking the mitochondrial targeting signal sequence as described in Brat et al. (2012).

Enzymes having BDH activity are encoded by genes: butA from Corynebacterium glutamicum, budC from Klebsiella pneumoniae 342, budC from Klebsiella oxytoca, EAE_{—}17490 from Enterobacter aerogenes, slaC from Serratia marcescens, bdh1 and bdh2 from Saccharomyces cerevisiae, bdhA from Bacillus subtilis subtilis 168, BMB171_C0587 from Bacillus thuriengensis or PPSC2_c3335 and PPSC2_c3890 from Bacillus polymyxa. Any BDH coding gene having at least 80-85%, preferentially 85-90%, more preferentially 90-95%, even more preferentially 96%, 97%, 98%, 99% sequence identity to any of those genes may be used.

In a preferred embodiment of the invention the BDH used is encoded by the gene budC from Klebsiella pneumoniae.

In another preferred embodiment the expressed BDH enzyme is encoded by the genes BDH1 and/or BDH2 from Saccharomyces cerevisiae.

More preferentially, in the bacterium of the invention BDH enzyme is encoded by the gene budC from Klebsiella pneumoniae whereas in the yeast of the invention BDH enzyme is encoded by the gene BDH1 from Saccharomyces cerevisiae.

In wild-type microorganism, conversion of acetolactate to diacetyl occurs spontaneously but slowly in presence of oxygen (Bassit et al., 1993).

In order to improve this conversion, gene encoding enzyme catalysing such conversion like ILV6 from S. cerevisiae or loxL2 from Lactobacillus sakei are overexpressed in the microorganisms of the invention. The lactate:oxygen 2-oxidoreductase encoded by loxL2 gene is evolved to obtain an enzyme having activity of conversion of acetolactate to diacetyl. The evolved enzymes are screened for their substrate specificity on acetolactate.

The 2,3-butanediol production pathway can be fully balanced under anaerobic conditions, however for the production of diacetyl oxygen is needed. Thus this problem can be solved by inactivating the respiratory chain to avoid the oxidation of NADH by aerobic respiration and to guarantee complete reduction of diacetyl to 2,3-butanediol, even in presence of oxygen.

It has been shown that in wild type Escherichia coli, the fermentation phenotype in presence of oxygen can be obtain by deleting the main cytochrome oxydase (cytochrome bo, bd-I and bd-II) (Portnoy et al., 2010).

In one embodiment of the invention, the cytochrome C oxydase of the yeast can be disrupted in order to obtain a fermentation phenotype despite the presence of oxygen. More precisely, we knock out one or several mitochondrial genes among COX1, COX2 and COX3. In combination with other modifications previously described this will promote the production of 2,3-butanediol via diacetyl, the sole fermentative pathway allowing redox balance.

In another aspect, the present invention relates to a method for producing 2,3-butanediol comprising the following steps: Step 1) preparing a strain modified with pathway 3 producing acetolactate, the precursor of the diacetyl by fermentation of the strain; Step 2) spontaneous decarboxylation of acetolactate in presence of oxygen by breaking the cells, for production of the diacetyl precursor of acetoin; Step 3) production of the 2,3-butanediol by enzymatic reaction process with the diacetyl precursor as a substrate and the converting enzyme 2,3-butanediol dehydrogenase.

In step 3, the bioconversion reaction transforms the diacetyl recovered from step 2 to acetoin, which is in turn enzymatically converted to 2,3-butanediol by the BDH enzyme overproduced in a different microorganism strain which is cultivated separately.

In a preferred embodiment, the recombinant bacterium of the invention expresses alsS and budC from Klebsiella pneumoniae and ILV6 gene from Saccharomyces cerevisiae, whereas endogenous genes encoding ALS, ALDC and BDH and poxB, ldhA, ackA and pta genes are deleted. Moreover, in this recombinant bacterium, expressions of aceE and aceF genes are attenuated.

In another preferred embodiment, the recombinant yeast of the invention expresses the ILV2 and BDH1 genes from Saccharomyces cerevisiae whereas ADH1, ADH3 and ADH5 endogenous genes from Saccharomyces cerevisiae are deleted.

Moreover, the recombinant yeast cell exhibits reduced activity of at least one enzyme catalysing the conversion of pyruvate to acetaldehyde in way to improve the conversion of pyruvate to acetolactate. Preferentially at least one gene chosen among PDC1, PDC5 or PDC6 is attenuated in the yeast of the invention. More preferentially the PDC1 gene is deleted. More preferentially the genes PDC1, PDC5 and PDC6 are deleted in the modified yeast.

Pathway 4

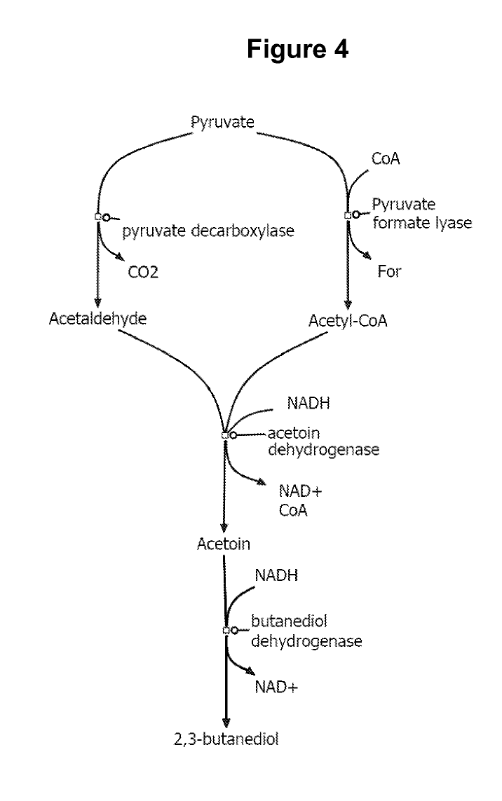

The fourth pathway of 2,3-butanediol biosynthesis (FIG. 4) includes steps of a) conversion of pyruvate to acetaldehyde by pyruvate decarboxylase (PDC) and to acetyl-CoA by pyruvate formate lyase (PFL), b) condensation of acetaldehyde and acetyl-CoA to acetoin by an acetoin dehydrogenase and c) conversion of acetoin to 2,3-butanediol by butanediol dehydrogenase (BDH).

The recombinant microorganism expresses the following enzymes:
a) a polypeptide catalysing the conversion of pyruvate to acetaldehyde and a polypeptide catalysing the conversion of pyruvate to acetyl-CoA, and
b) a polypeptide catalysing the condensation of acetyl-CoA and acetaldehyde to acetoin, and
c) a butanediol dehydrogenase (BDH).

In one embodiment, the recombinant microorganism of the invention expresses genes encoding polypeptides having activity of pyruvate decarboxylase (PDC), pyruvate formate lyase (PFL), acetoin dehydrogenase and butanediol dehydrogenase (BDH).

As described above, in yeast the PDC enzymes are encoded by the genes PDC1, PDC5 and PDC6. In a preferred embodiment of the invention at least one of the PDC1, PDC5, PDC6 genes of Saccharomyces cerevisiae are overexpressed in the recombinant bacterium of the invention. More preferentially, PDC1 from Saccharomyces cerevisiae is overexpressed in the recombinant bacterium.

In another embodiment of the invention, the recombinant yeast overexpresses the pflA and/or pflB genes of Escherichia coli. As in pathway 2 formate may be purified or disintegrated.

Enzymes having BDH activity are encoded by genes: butA from Corynebacterium glutamicum, budC from Klebsiella pneumoniae 342, budC from Klebsiella oxytoca, EAE_{—}17490 from Enterobacter aerogenes, slaC from Serratia marcescens, BDH1 and BDH2 from Saccharomyces cerevisiae, bdhA from Bacillus subtilis subtilis 168, BMB171_C0587 from Bacillus thuriengensis or PPSC2_c3335 and PPSC2_c3890 from Bacillus polymyxa. Any BDH coding gene having at least 80-85%, preferentially 85-90%, more preferentially 90-95%, even more preferentially 96%, 97%, 98%, 99% sequence identity to any of those genes may be used.

In a preferred embodiment of the invention the BDH used is encoded by the gene budC from Klebsiella pneumoniae.

In another preferred embodiment the expressed BDH enzyme is encoded by the genes BDH1 and BDH2 from Saccharomyces cerevisiae.

More preferentially, in the bacterium of the invention BDH enzyme is encoded by the gene budC from Klebsiella pneumoniae whereas in the yeast of the invention BDH enzyme is encoded by the gene BDH1 from Saccharomyces cerevisiae.

Acetoin dehydrogenase activity is catalysed for example by the protein AcoA and AcoB encoded by genes acoA and acoB from Bacillus subtilis, AcoB encoded by the gene acoB from Pseudomonas putida, ButA encoded by the gene butA from Streptococcus gordonii, AcoA, AcoB and AcoC encoded by genes acoA, acoB and acoC from Pelobacter carbinolicus or by AcoC encoded by the gene acoC from Bacillus licheniformis. The different proteins or genes may be modified in order to improve the activity of condensation of acetaldehyde and acetyl-CoA to acetoin. Preferably, acoA and acoB from Bacillus subtilis are overexpressed in the microorganism of the invention.

In a preferred embodiment, the recombinant bacterium of the invention overexpresses PDC1 gene from Saccharomyces cerevisiae, pflB gene from Escherichia coli, acoA and acoB genes from Bacillus subtilis and budC gene from Klebsiella pneumoniae, whereas endogenous genes encoding ALS, ALDC and BDH and poxB, ldhA, ackA and pta genes are deleted.

In another preferred embodiment, the recombinant yeast of the invention expresses the PDC1 gene from Saccharomyces cerevisiae and overexpressed pflA and pflB genes from Escherichia coli, acoA and acoB genes from Bacillus subtilis and BDH1 gene from Saccaromyces cerevisiae, whereas ADH1, ADH3, ADH5 and ILV2 endogenous genes from Saccharomyces cerevisiae are deleted.

Pathway 5

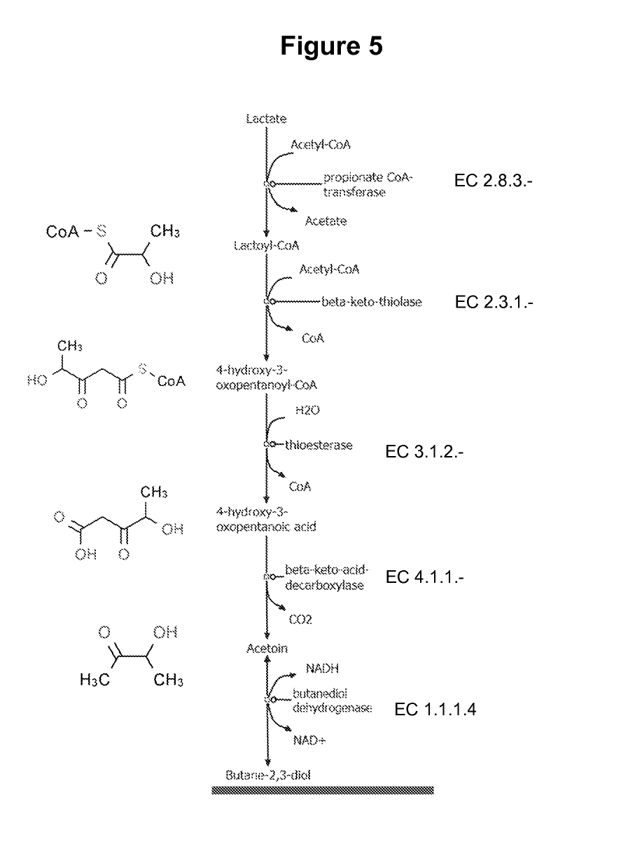

The fifth pathway of 2,3-butanediol biosynthesis (FIG. 5) includes steps of conversion of lactate to lactoyl-CoA by propionate-CoA transferase, conversion of lactoyl-CoA to 4-hydroxy-3-oxopentanoyl-CoA by beta-keto-thiolase, conversion of 4-hydroxy-3-oxopentanoyl-CoA to 4-hydroxy-3-oxopentanoic acid by thioesterase, conversion of 4-hydroxy-3-oxopentanoic acid to acetoin by beta-keto-acid-decarboxylase and conversion of acetoin to 2,3-butanediol by butanediol dehydrogenase (BDH).

In this pathway, the lactate is obtained by conversion of pyruvate into lactate by the lactate dehydrogenase activity.

In one embodiment, the recombinant microorganism of the invention expresses genes encoding polypeptides having activity of propionate-CoA transferase, beta-keto-thiolase, thioesterase, beta-keto-acid-decarboxylase and butanediol dehydrogenase (BDH).

Enzymes having propionate-CoA transferase activity are for example enzyme encoded by the pct gene from Listeria monocytogenes_F2365, the pct gene from Listeria welshimeri, the pct gene from Roseobacter denitrificans, the pct gene from Ralstonia eutropha, the pct gene from Rhizobium leguminosarum or the ydiF gene from Escherichia coli_APEC.

Enzyme having beta-keto-thiolase activity are for example enzyme encoded by the paaJ gene from Escherichia coli, the pcaF gene from Pseudomonas knackmussii, the phaD or pcaF gene from Pseudomonas putida or the paaE from Pseudomonas fluorescens.

Enzymes having thioesterase activity are for example enzyme encoded by the ACOT12 gene from Bos taurus, the yneP gene from Bacillus subtilis, the ypch01226 gene from Rhizobium etli, the hibch gene from Mus musculus, the hibch gene from Ratus norvegicus.

Enzymes having beta-keto-acid-decarboxylase activity are for example enzyme coded by the kivD gene from Lactococcus lactis, the pdc gene from Clostridium acetobutylicum or by the pdc2 gene from Pichia stipitis.

Enzymes having BDH activity are encoded by genes: butA from Corynebacterium glutamicum, budC from Klebsiella pneumoniae 342, budC from Klebsiella oxytoca, EAE_{—}17490 from Enterobacter aerogenes, slaC from Serratia marcescens, BDH1 and BDH2 from Saccharomyces cerevisiae, bdhA from Bacillus subtilis subtilis 168, BMB171_C0587 from Bacillus thuriengensis or PPSC2_c3335 and PPSC2_c3890 from Bacillus polymyxa.

Any protein encoding a gene having at least 80-85%, preferentially 85-90%, more preferentially 90-95%, even more preferentially 96%, 97%, 98%, 99% sequence identity to any of those genes may be used.

Pathway 6

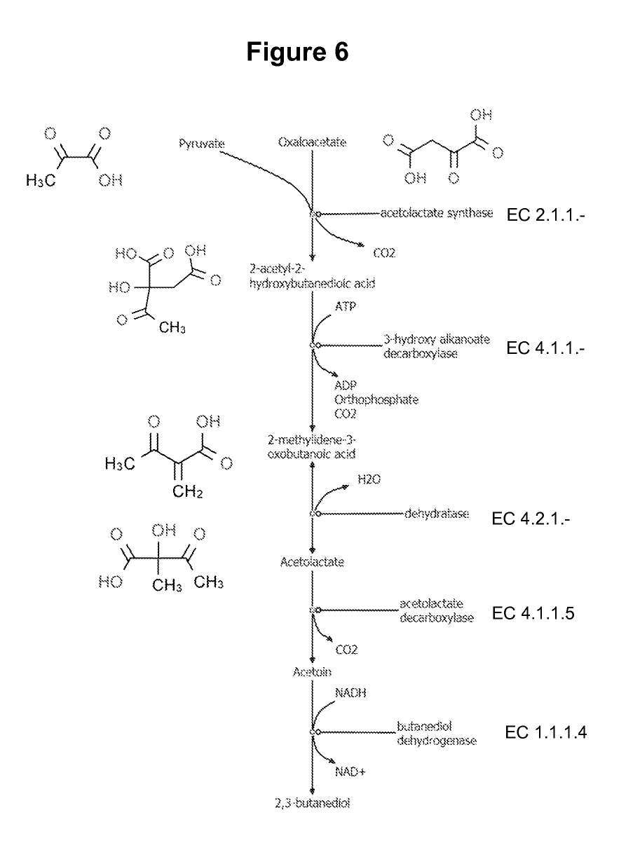

The sixth pathway of 2,3-butanediol biosynthesis (FIG. 6) includes steps of condensation of pyruvate and oxaloacetate to 2-acetyl-2-hydroxybutanedioic acid by acetolactate synthase (ALS), conversion of 2-acetyl-2-hydroxybutanedioic acid to 2-methylidene-3-oxobutanoic acid by 3-hydroxy alkanoate decarboxylase, conversion of 2-methylidene-3-oxobutanoic acid to acetolactate by a dehydratase, conversion of acetolactate to acetoin by acetolactate decarboxylase (ALDC) and conversion of acetoin to 2,3-butanediol by butanediol dehydrogenase (BDH).

In one embodiment, the recombinant microorganism of the invention expresses genes encoding polypeptides having activity of acetolactate synthase (ALS), 3-hydroxy alkanoate decarboxylase, dehydratase, acetolactate decarboxylase (ALDC), butanediol dehydrogenase (BDH).

Enzymes having acetolactate synthase (ALS) activity for the condensation of pyruvate and oxaloacetate to 2-acetyl-2-hydroxybutanedioic acid are for example enzyme encoded by the ilvB gene from Escherichia coli, the ilvH gene from Bacillus cereus, the ilvH gene from Corynebacterium glutamicum, the csr1 gene from Arabidopsis thaliana.

Enzymes having 3-hydroxy alkanoate decarboxylase activity are for example enzymes encoded by the mvaD gene from Enterococcus faecalis, the yeaH gene from Lactococcus lactis, the NCUO2127.1 gene from Neurospora crassa or the MVD1 from Schizosaccharomyces pombe.

Enzymes having dehydratase activity for the conversion of 2-methyldene-3-oxobutanoic acid to acetolactate are for example enzymes encoded by the CSE45_{—}3601 gene from Citreicella sp. SE45, the caiD gene from Bordetella parapertussis, the abfD gene from Porphyromonas gingivalis.

Enzymes having ALDC activity are for example encoded by genes budA from Klebsiella pneumoniae, budA from Klebsiella oxytoca, budA from Enterobacter aerogenes, salA from Serratia marcescens, alsD from Bacillus subtilis subtilis 168, alsD from Bacillus thuriengiensis or PPSC2_c2281 from Bacillus polymyxa.

Enzymes having BDH activity are encoded by genes: butA from Corynebacterium glutamicum, budC from Klebsiella pneumoniae 342, budC from Klebsiella oxytoca, EAE_{—}17490 from Enterobacter aerogenes, slaC from Serratia marcescens, BDH1 and BDH2 from Saccharomyces cerevisiae, bdhA from Bacillus subtilis subtilis 168, BMB171_C0587 from Bacillus thuriengensis or PPSC2_c3335 and PPSC2_c3890 from Bacillus polymyxa.

Any protein encoding a gene having at least 80-85%, preferentially 85-90%, more preferentially 90-95%, even more preferentially 96%, 97%, 98%, 99% sequence identity to any of those genes may be used.

### Optimisations of 2,3-Butanediol Production

In another aspect of the invention, the recombinant microorganism is further modified to improve the availability of pyruvate, by attenuating the degradation pathway of pyruvate in other product than 2,3-butanediol.

In yeast, attenuation lactate production from pyruvate, is achieved by deleting in the recombinant yeast of the invention at least one gene chosen among DLD1, CYB2, DLD3 and DLD2. Preferably the genes DLD1, CYB2, DLD3 and DLD2 are deleted in the modified yeast.

For attenuating fumarate and succinate production from pyruvate, the FUM1 gene is attenuated in the modified yeast. The gene FUM1 seems to be essential for the yeast, thus attenuation is preferred in this embodiment.

For attenuating glycerol production from pyruvate, at least one gene chosen among HOR2, RHR2, GPD1, GPD2, GUT1 and TDA10 is deleted in the recombinant yeast of the invention. Preferably the genes HOR2, RHR2, GPD1, GPD2, GUT1 and TDA10 are deleted in the modified yeast.

In bacterium, optimization of pyruvate availability is achieved by overexpressing, in the bacterium of the invention, at least one gene involved in pyruvate biosynthesis pathway, chosen among genes coding for phosphoglycerate mutase (gpmA and pgmI genes from Escherichia coli or homologous gene), enolase (eno from Escherichia coli or homologous gene) or pyruvate kinase (pykA and pykF genes from Escherichia coli or homologous gene). Alternatively or in combination, at least one gene involved in pyruvate degradation pathway is attenuated. This gene is chosen among pyruvate oxidase (poxB from Escherichia coli or homologous gene), phosphate acetyltransferase (pta from Escherichia coli or homologous gene), acetate kinase (ackA from Escherichia coli or homologous gene) or lactate dehydrogenase (lldD, ldhA or did from Escherichia coli or homologous gene).

Under fermentative conditions (anaerobia, microaerobia or the presence of large amounts of sugar), yeast that will produce 2.3,-butanediol will only be able to oxidise 50% of all NADH produced during glycolysis. These electrons need to be transferred to other artificial electron acceptors.

In one embodiment of the invention, these electron acceptors can be oxidized forms of nitrogen, such as nitrate, nitrite or nitrous oxide, or nitrogen itself. Certain fungi are capable of reducing these nitrogen compounds to ammonia under anaerobic or microaerobic conditions by a process called “ammonia fermentation”, as described in Zhou et al. (2002) or Takasaki et al. (2004). These low oxygen conditions present an advantage for industrial production since cost of aeration can be eliminated. Thus either the BDO production pathway as described in patent application WO2011041426 will be implemented into an organism capable of performing “ammonia fermentation”, or preferentially the fermentation pathway will be transferred into a BDO producing yeast strain. Under certain conditions it may be necessary to express a constitutive ATPase to reduce the amount of ATP in excess in the cell.

In another embodiment of the invention, NADH-dependant glyceraldehyde-3-phosphate dehydrogenase is replaced by an NADPH-dependant enzyme. In this case the BDO producing organism will produce an excess of NADPH that in turn can be used via assimilatory nitrate reduction, requiring the introduction of nitrate reductase into certain yeast strains that lack this enzyme.

In another embodiment of the invention, other electron accepting substrates can be added. These can be inorganic substrates, such as sulphate and oxidised iron or organic substrate such as for example mannose that will be reduced to mannitol. NADH or NADPH excess can also be used to transform oxidized precursor molecules into reduced products. In this way the excess reducing power can be utilized to generate valuable co-products that can be recovered from the fermentation broth.

It is preferred that the engineered biosynthetic pathway provides at least partial redox balance to the cell. At least partial redox balance may be achieved, for example, by including an enzyme in the engineered biosynthetic pathway that requires NADH for its activity. Utilizing NADH balances production of NADH during conversion of glucose to pyruvate. In wild type cells NADH is utilized in conversion of glucose to glycerol, and in production of ethanol from pyruvate. The present adh-production yeast cells have unbalanced NADH due to disruption of ethanol production. Any method of increasing NADH-dependent enzyme activity in the present production host cell may be used in balancing redox. In addition to including a NADH-dependent enzyme in the biosynthetic pathway, methods include expressing an enzyme that requires NADH but that is not part of the engineered pyruvate-utilizing biosynthetic pathway. A redox-balancing NADH-dependent enzyme may be expressed from a heterologous gene.

Alternatively, expression of an endogenous gene encoding an NADH-dependent enzyme may be increased to provide increased NADH-dependent enzyme activity.

In the invention, biosynthesis pathways 1, 5 and 6 are partially redox balanced but pathways 2, 3 and 4 are totally redox balanced.

### Strain Optimization to Enhance the Production of 2,3-Butanediol

Sugar Import—Improvement of Glucose Import

Like bacteria, yeasts consume mono- and disaccharides preferentially to other carbohydrates. In yeasts however, the major sugar transporters work by an energy-independent, facilitated diffusion mechanism. These so-called hexose (Hxt) and galactose (Gal2) transporters naturally exhibit different affinities and specificities for their substrates. They are abundant in S. cerevisiae but much less so in other industrial important yeast strains including Kluveromyces lactis and Pichia stipitis (Boles & Hollenberg, 1997; Wieczorke et al., 1999).

One way to improve the glucose import into the strain producing 2,3-butanediol is to overexpress the genes encoding the hexose and galactose transporters or to modify the genes involved in their complex regulatory pathway, Snf3/Rgt2-Rgt1.

In an embodiment of the invention the recombinant yeast overexpresses at least one gene of the following: HXT1, HXT2, HXT4, HXT5, HXT7 and GAL2.

Sugar Import—ATP Consumption

As identified by Larsson and co-workers in 1997, the glycolytic flux is conditionally correlated with ATP concentration in S. cerevisiae. ATP has a strong negative effect on glycolytic activity affecting several of the glycolytic enzymes. However, the main targets for ATP inhibition were phosphofructokinase and pyruvate kinase. Other potential candidates as enzymes susceptible to ATP inhibition included hexokinase and enolase (Larsson et al., 1997 and 2000).

In an embodiment of the invention, the recombinant microorganism producing 2,3-butanediol is further modified to consume its excess of ATP, by overexpressing the soluble part of an ATPase enzyme. This can be accomplished by either overexpressing endogenous genes ATP1, ATP2 and ATP3 of the yeast strain or by introducing heterologous genes, as for instance atpAGD from E. coli.

In another embodiment of the invention, glucose import is improved by expressing at least one desensitized allele of genes known to be inhibited by an excess of ATP such as: PFK1, PFK2, PYK1, PYK2, HXK1, HXK2, YLR446W, GLK1, ENO1 and ENO2.

Sugar Import—Improvement of C5 Sugar Import

The import of pentoses by recombinant microorganism is a major issue for industrial process since C5 sugars are major constituents of hydrolysed lignocellulosic biomass. Native strains of S. cerevisiae, like many other types of yeast, are unable to utilize either xylose or arabinose as fermentative substrates (Hahn-Hagerdal et al., 2007; Jin et al., 2004). Interestingly, it is able to uptake xylose even though the sugar is not a natural substrate (Hamacher et al., 2002).

S. cerevisiae GAL2, HXT1, HXT2, HXT4, HXT5, and HXT7 catalyze the uptake of xylose because they have a broad substrate specificity (Hamacher et al., 2002; Saloheimo et al., 2007; Sedlak & Ho 2004). However, their affinity for xylose is much lower than that for glucose and the xylose uptake by the transporters is strongly inhibited by glucose (Saloheimo et al., 2007).

Several changes are needed to obtain a strain able to grow and consume xylose and/or arabinose. These different modifications are a part of the invention.

Overexpression of Heterologous Xylose Transporters

In order to improve the xylose and arabinose uptake, the recombinant 2,3-BDO producer strain has to be modified to express heterologous genes coding for xylose or arabinose transporters. For example, genes GXF1, SUT1 and AT5g59250 from Candida intermedia, Pichia stipitis and Arabidopsis thaliana, respectively, are overexpressed to improve xylose utilization by the yeast (Runquist et al., 2010).

Overexpression of Pathways Involved in the Metabolism of Xylose and Arabinose

Yeast strains are able to take up xylose even though the sugar is not a natural substrate. Even though genes for xylose assimilation are present in S. cerevisiae they are not expressed at a sufficient level to enable significant sugar assimilation. Thus genetic modifications are necessary to improve the assimilation of pentose sugars. All enzymes that allow the transformation of xylose or arabinose to xylitol need to be enhanced as well as the enzymes which convert xylitol in xylulose, and xylulose into xylulose-5-phosphate.

Either, the homologous genes from the xylose and arabinose pathways have to be overexpressed or heterologous genes from bacteria have to be overexpressed.

In one embodiment of the invention the xylose uptake and its assimilation by the strain are improved by overexpressing for example:
1) Genes XYL1 or GRE3 coding the aldolase reductase of P. stipitis and S. cerevisiae, respectively, associated to overexpression of XYL2 encoding the xylitol dehydrogenase from P. stipitis, combined with the overexpression of genes XKS1 or XYL3 encoding the xylulokinase from S. cerevisiae and P. stipitis, respectively,
2) The gene xylA encoding a xylose isomerase from bacteria or Piromyces associated to the overexpression of genes XKS1 or XYL3 encoding the xylulokinase from S. cerevisiae and P. stipitis, respectively.

In another embodiment of the invention, arabinose uptake and its assimilation by the strain are improved by overexpressing for example:
1) Homologous genes XYL1 or GRE3 coding the aldolase reductase of P. stipitis and S. cerevisiae, respectively, associated to ladI encoding the L-arabinitol 4-hydrogenase and lxr1 encoding a L-xylulose reductase from Trichoderma reesei, in combination with the overexpression of XYL2 encoding the xylitol dehydrogenase from P. stipitis, and in addition the overexpression of genes XKS1 or XYL3 encoding the xylulokinase from S. cerevisiae and P. stipitis, respectively,
2) Heterologous genes araA and araB encoding bacterial arabinose isomerase and ribulose kinase.

Optimization of the Pentose Phosphate Pathway

This can be done by overexpressing at least one gene belonging to the non oxidative pentose phosphate pathway; TAL1, TKL1, RKL1 and RPE1 from the yeast strain.

Optimization of the Availability of NAPDH Cofactors Required by the Enzymes Involved in the Metabolism of C5-Sugars

This is attained by expressing the transhydrogenases of E. coli in the yeast strain. The genes udhA and or pntAB from E. coli will be overexpressed in the producer strain.

The carbon source used in this invention comprises hexoses or pentoses or a mixture thereof.

### Culture Conditions

The invention is also related to a method of production of 2,3-butanediol (BDO) comprising the following steps:
providing a recombinant microorganism as previously described,
cultivating the recombinant microorganism in a culture medium containing a source of carbon, and
recovering the 2,3-butanediol.

Typically, microorganisms of the invention are grown at a temperature in the range of about 20° C. to about 37° C. in an appropriate medium.

Suitable growth media for yeast are common commercially prepared media such as broth that includes yeast nitrogen base, ammonium sulfate, and dextrose as the carbon/energy source) or YPD Medium, a blend of peptone, yeast extract, and dextrose in optimal proportions for growing most. Other defined or synthetic growth media may also be used and the appropriate medium for growth of the particular microorganism will be known by one skilled in the art of microbiology or fermentation science.

The term an ‘appropriate culture medium’, as used herein for bacterium, refers to a medium (e.g., a sterile, liquid media) comprising nutrients essential or beneficial to the maintenance and/or growth of the cell such as carbon sources or carbon substrate, nitrogen sources, for example, peptone, yeast extracts, meat extracts, malt extracts, urea, ammonium sulfate, ammonium chloride, ammonium nitrate and ammonium phosphate; phosphorus sources, for example, monopotassium phosphate or dipotassium phosphate; trace elements (e.g., metal salts), for example magnesium salts, cobalt salts and/or manganese salts; as well as growth factors such as amino acids, vitamins, growth promoters, and the like.

As an example of known culture media for E. coli, the culture medium can be of identical or similar composition to an M9 medium (Anderson, 1946), an M63 medium (Miller, 1992) or a medium such as defined by Schaefer et al. (1999).

Suitable pH ranges for the fermentation are between pH 3.0 to pH 7.5, where pH 4.5 to pH 6.5 is preferred as the initial condition.

Fermentations may be performed under aerobic or anaerobic conditions, where anaerobic or micro-aerobic conditions are preferred.

The amount of product in the fermentation medium can be determined using a number of methods known in the art, for example, high performance liquid chromatography (HPLC) or gas chromatography (GC).

The present process may employ a batch method of fermentation. A classical batch fermentation is a closed system where the composition of the medium is set at the beginning of the fermentation and not subject to artificial alterations during the fermentation. Thus, at the beginning of the fermentation the medium is inoculated with the desired organism or organisms, and fermentation is permitted to occur without adding anything to the system. Typically, however, a “batch” fermentation is batch with respect to the addition of carbon source and attempts are often made at controlling factors such as pH and oxygen concentration. In batch systems the metabolite and biomass compositions of the system change constantly up to the time when the fermentation is stopped. Within batch cultures cells progress through a static lag phase to a high growth log phase and finally to a stationary phase where growth rate is diminished or halted. If untreated, cells in the stationary phase will eventually die. Cells in log phase generally are responsible for the bulk of production of end product or intermediate.

A Fed-Batch system may also be used in the present invention. A Fed-Batch system is similar to a typical batch system with the exception that the carbon source substrate is added in increments as the fermentation progresses. Fed-Batch systems are useful when catabolite repression (e.g. glucose repression) is apt to inhibit the metabolism of the cells and where it is desirable to have limited amounts of substrate in the media. Measurement of the actual substrate concentration in Fed-Batch systems is difficult and is therefore estimated on the basis of the changes of measurable factors such as pH, dissolved oxygen and the partial pressure of waste gases such as CO₂.

Fermentations are common and well known in the art and examples may be found in Sunderland et al., (1992), herein incorporated by reference. Although the present invention is performed in batch mode it is contemplated that the method would be adaptable to continuous fermentation.

Continuous fermentation is an open system where a defined fermentation medium is added continuously to a bioreactor and an equal amount of conditioned media is removed simultaneously for processing. Continuous fermentation generally maintains the cultures at a constant high density where cells are primarily in log phase growth.

Continuous fermentation allows for the modulation of one factor or any number of factors that affect cell growth or end product concentration. For example, one method will maintain a limiting nutrient such as the carbon source or nitrogen level at a fixed rate and allow all other parameters to vary. In other systems a number of factors affecting growth can be altered continuously while the cell concentration, measured by media turbidity, is kept constant. Continuous systems strive to maintain steady state growth conditions and thus the cell loss due to the medium being drawn off must be balanced against the cell growth rate in the fermentation. Methods of modulating nutrients and growth factors for continuous fermentation processes as well as techniques for maximizing the rate of product formation are well known in the art of industrial microbiology.

It is contemplated that the present invention may be practiced using either batch, fed-batch or continuous processes and that any known mode of fermentation would be suitable. Additionally, it is contemplated that cells may be immobilized on a substrate as whole cell catalysts and subjected to fermentation conditions for production.

### Purification of 2,3-Butanediol

According to a specific aspect of the invention, the fermentative production of 2,3-butanediol comprises a step of isolation of the 2,3-butanediol from the culture medium. Recovering the 2,3-butanediol from the culture medium is a routine task for a man skilled in the art. It may be achieved by a number of techniques well known in the art including but not limiting to distillation, gas-stripping, pervaporation or liquid extraction. The expert in the field knows how to adapt parameters of each technique dependant on the characteristics of the material to be separated.

Distillation may involve an optional component different from the culture medium in order to facilitate the isolation of 2,3-butanediol by forming azeotrope and notably with water. This optional component is an organic solvent such as cyclohexane, pentane, butanol, benzene, toluene, trichloroethylene, octane, diethylether or a mixture thereof.

Gas stripping is achieved with a stripping gas chosen among helium, argon, carbon dioxide, hydrogen, nitrogen or mixture thereof.

Liquid extraction is achieved with organic solvent as the hydrophobic phase such as pentane, hexane, heptane, dodecane.

The purification conditions may be specifically adapted to the downstream transformation of 2,3-BDO to 1,3-butadiene, including keeping several co-products in the partially purified 2,3-BDO.

### EXAMPLES

The present invention is further defined in the following examples. It should be understood that these examples, while indicating preferred embodiments of the invention, are given by way of illustration only. From above disclosure and these examples, the man skilled in the art can make various changes of the invention to adapt it to various uses and conditions without modify the essentials means of the invention.

In particular, examples show recombinant Escherichia coli (E. coli) strains and Saccharomyces cerevisia strains (S. cerevisiae), but the genetic modifications described in here can easily be performed on other microorganisms of the same family.

E. coli belongs to the Enterobacteriaceae family, which comprises members that are Gram-negative, rod-shaped, non-spore forming and are typically 1-5 μm in length. Most members have flagella used to move about, but a few genera are non-motile. Many members of this family are a normal part of the gut flora found in the intestines of humans and other animals, while others are found in water or soil, or are parasites on a variety of different animals and plants. E. coli is one of the most important model organisms, but other important members of the Enterobacteriaceae family include Klebsiella, in particular Klebsiella terrigena, Klebsiella planticola or Klebsiella oxytoca, and Salmonella.

S. cerevisiae is a model organism and belongs to the Saccharomycetaceae family, which comprises members that are spherical, elliptical or oblong acuminate cells with an asexual reproduction by budding and are typically 5-10 μm in diameter. Many members are used for industrial production due to their fermentative capacity. S. cerevisiae is one of the most important model organisms, but other important members of the Saccharomycetaceae family include Pichia, Candida and Kluyveromyces, in particular Kluyveromyces lactis, Kluyveromyces marxianus and Pichia angusta.

TABLE 1
Genotype and corresponding number of intermediates strains and producing
strains described in the following examples.

| Strain number | Génotype |
|---|---|
| 1 (MG1655) | F-lambda-ilvG-rfb-50 rph-1 |
| 2 | MG1655 ΔpoxB ΔldhA |
| 3 | MG1655 ΔpoxB ΔldhA ΔackA-pta::Km |
| 4 | MG1655 ΔpoxB ΔldhA ΔackA-pta::Km pCL1920-Ptrc01/E02/RBS01*2-pdc1sc |
| | pBBR1MCS5-Ptrc01/E02/RBS01*2-budCkp-Ptrc01/RBS01*2-pflB |
| 5 | MG1655 ΔpoxB ΔldhA ΔackA-pta::Km (pCL1920-Ptrc01/E02/RBS01*2-pdc1sc) |
| | (pBBR1MCS5-Ptrc01/E02/RBS01*2-budCkp-Ptrc01/RBS01*2-pflB-Ptrc01/RBS01*2- |
| | mhpF-TT07) |
| 6 | MG1655 ΔpoxB ΔldhA Ptrc157-aceEF::Km |
| 7 | MG1655 ΔpoxB ΔldhA Ptrc157-aceEF::Km (pBBR1MCS5-Ptrc01/E02/RBS01*2-budCkp- |
| | Ptrc01/RBS01*2-alsSkpO1ec-TT07) (pCL1920-Ptrc01/E02/RBS01*2-ilv6scO1ec-TT07) |
| 8 | MG1655 ΔpoxB ΔldhA ΔackA-pta::Km Ptrc01/E06/RBS01*2-pflA::Cm |
| 9 | MG1655 ΔpoxB ΔldhA ΔackA-pta::Km Ptrc01/E06/RBS01*2-pflA::Cm (pCL1920- |
| | Ptrc01/E02/RBS01*2-pdc1sc) (pBBR1MCS5-Ptrc01/E02/RBS01*2-budCkp- |
| | Ptrc01/RBS01*2-pflB-UTR-acoABbs-TT07) |
| 10 (CEN.PK2-1C) | MATa ura3-52 trp1-289 leu2-3,112 his3Δ 1 MAL2-8^{c} SUC2 |
| 11 | MATa ura3-52 trp1-289 leu2-3,112 his3Δ 1 MAL2-8^{c} SUC2 Δadh1 Δadh3 Δadh5::Gn |
| 12 | MATa ura3-52 trp1-289 leu2-3,112 his3Δ 1 MAL2-8^{c} SUC2 Δadh1 Δadh3 |
| | Δadh5::Gn Δilv2::Hg |
| 13 | MATa ura3-52 trp1-289 leu2-3,112 his3Δ 1 MAL2-8^{c} SUC2 Δadh1 Δadh3 |
| | Δadh5::Gn Δilv2::Hg (p424-Phxt7-bdh1-TTcyc1)(p426-Phxt7-mhpFec-TTcyc1) (p423- |
| | Phxt7-pflAec-TTcyc1-Ppgk1-pflBec-TTpdc1) |
| 14 | MATa ura3-52 trp1-289 leu2-3,112 his3Δ 1 MAL2-8^{c} SUC2 Δadh1 Δadh3 |
| | Δadh5::Gn Δilv2::Hg (p424-Phxt7-bdh1-TTcyc1) (p426-Phxt7-acoAbs-TTcyc1-Ppgk1- |
| | acoBbs-TTpdc1) (p423-Phxt7-pflAec-TTcyc1-Ppgk1-pflBec-TTpdc1) |

### Protocols:

The E. coli strains engineered to produce 2.3BDO were generated using procedures described in patent application WO2010/076324. The gene disruption in specified chromosomal locus was carried out by homologous recombination as described by Datsenko & Wanner (2000). The antibiotic resistant cassette can be amplified on pKD3, pKD4, pKD13 or any other plasmid containing another antibiotic resistant gene surrounded by FRT sites. Chromosomal modifications were transferred to a given E. coli recipient strain by P1 transduction.

The yeast strains engineered to produce 2.3BDO were generated using procedures described by Baudin et al. (1993) and Wach et al. (1994). Chromosomal deletions used PCR-generated deletion strategy to replace yeast open reading frame of interest from its start- to stop-codon with a cassette resistance module (KanMX conferring geneticin resistance or Hph (hygromycin B phosphotransferase) conferring hygromycin B resistance). Plasmid constructions were done by homologous recombination as described by Ma et al. (1987) and Raymond et al (1999).

### Example 1

### Construction of Strain 5: Overexpression of Pyruvate Decarboxylase, Pyruvate Formate Lyase, Acetaldehyde Dehydrogenase and Butanediol Dehydrogenase Genes in E. coli

### Construction of Strain 2

In order to prevent the accumulation of acetate and the consumption of pyruvate during E. coli cells culture, poxB gene encoding pyruvate oxidase was deleted by using primers DpoxB-F (SEQ ID No 01) and DpoxB-R (SEQ ID No 02).

In order to prevent the accumulation of lactate and the consumption of pyruvate during E. coli cells culture, ldhA gene encoding lactate dehydrogenase was deleted by using primers D1dhA-F (SEQ ID No 03) and D1dhA-R (SEQ ID No 04).

Both deletions were introduced in MG1655 using homologous recombination method in Escherichia coli (Datsenko & Wanner (2000)). This method allowed the combination of multiple genetic modifications using different antibiotic resistance genes which can be removed. The resulting strain MG1655 ΔpoxB ΔldhA was called strain 2 (Table 1).

### Construction of Strain 3

In order to prevent the accumulation of acetate during the culture, the deletion of ackA-pta transcription unit was performed. The acetate production by AckA-Pta pathway contains two enzymes: the phosphotransacetylase (pta) that reversibly converts acetyl-CoA, and inorganic phosphate to acetyl phosphate and CoA, and the acetate kinase (ackA) that reversibly converts acetyl phosphate and ADP to acetate and ATP.

The ackA-pta transcription unit was deleted by using primers DackA-pta-F (SEQ ID No 05) and DackA-pta-R (SEQ ID No 06) and introduced into strain 2 by P1 transduction. The validated strain MG1655 ΔpoxB ΔldhA ΔackA-pta::Km was called strain 3 (Table 1)

### Construction of Strain 4

Construction of Plasmid pCL1920-Ptrc01/E02/RBS01*2-Pdc1Sc

The plasmid pCL1920-Ptrc01/E02/RBS01*2-PDC1 sc was derived from pCL1920 (Lerner & Inouye, 1990, NAR 18, 15 p 4631), PDC1sc gene encoding the pyruvate decarboxylase from Saccharomyces cerevisiae S288C which expression was driven by a constitutive Ptrc promoter and an optimized RBS sequence. The PDC1sc gene was PCR-amplified with primers Ptrc01/E02/RBS01*2-pdc1sc F (SEQ ID No 07) and pdc1sc R (SEQ ID No 08) using Saccharomyces cerevisiae reference strain S288C genomic DNA and cloned between the SacI and BamHI restriction sites of pCL1920 plasmid. The resulting plasmid was called pCL1920-Ptrc01/E02/RBS01*2-pdc1sc.

Construction of Plasmid pBBR1MCS5-Ptrc01/E02/RBS01*2-budCkp-Ptrc01/RBS01*2-pflB

The pBBR1MCS5-Ptrc01/E02/RBS01*2-budCkp-Ptrc01/RBS01*2-pflB plasmid is derived from pBBR1MCS5 plasmid (M. E. Kovach, (1995), Gene 166:175-176), budCkp synthetic gene encoding butanediol dehydrogenase and pflB gene from E. coli encoding the pyruvate formate lyase. In this plasmid, expression of the budCkp synthetic gene and pflB gene were driven by independents constitutive trc promoters and optimized RBS sequences.

Synthetic Gene budC

A budC synthetic gene from Klebsiella pneumoniae MGH78578 coding for the butanediol dehydrogenase (acetoin reductase) was synthesized by Life Technologies (SEQ ID No 54). The construct was cloned into the supplier's pM vector and verified by sequencing.

Construction of pBBR1MCS5-Ptrc01/E02/RBS01*2-budCkp Plasmid

The Ptrc01/E02/RBS01*2-budCkp fragment was PCR-amplified with primers Ptrc01/E02/RBS01*2-budCkp F (SEQ ID No 09) and budCkp R (SEQ ID No 10) using the pMA-T-budCkp vector provided by the supplier and cloned between the SacI and XbaI sites of the pBBR1MCS5 plasmid. The resulting plasmid was called pBBR1MCS5-Ptrc01/E02/RBS01*2-budCkp.

Construction of the Plasmid pBBR1MCS5-Ptrc01/E02/RBS01*2-budCkp-Ptrc01/RBS01*2-pflB

In order to increase the acetyl-CoA pool in the cell, pflB gene from E. coli was over-expressed. This enzyme catalyses by an oxygen-sensitive radical-based reaction the acetyl-CoA synthesis from pyruvate without ATP consumption.

“Ptrc01/RBS01*2-pflB” fragment was PCR-amplified with primers Ptrc01-pflB-SpeI F (SEQ ID No 11) and pflB-EcoRI R (SEQ ID No 12) using E. coli MG1655 genomic DNA and cloned between the SpeI and EcoRI sites of the pBBR1MCS5-Ptrc01/E02/RBS01*2-budCkp plasmid described above. The resulting plasmid was called pBBR1MCS5-Ptrc01/E02/RBS01*2-budCkp-Ptrc01/RBS01*2-pflB.

### Construction of Strain 4

The pCL1920-Ptrc01/E02/RBS01*2-pdc1sc and pBBR1MCS5-Ptrc01/E02/RBS01*2-budCkp-Ptrc01/RBS01*2-pflB were introduced by electroporation into strain 3 (Table 1). The resulting strain MG1655 ΔpoxB ΔldhA ΔackA-pta::Km (pCL1920-Ptrc01/E02/RBS01*2-pdc1sc) (pBBR1MCS5-Ptrc01/E02/RBS01*2-budCkp-Ptrc01/RBS01*2-pflB) was called strain 4 (Table 1).

### Construction of Strain 5

Construction of Plasmid pBBR1MCS5-Ptrc01/E02/RBS01*2-budCkp-Ptrc01/RBS01*2-pflB-Ptrc01/RBS01*2-mhpF-TT07

The plasmid pBBR1MCS5-Ptrc01/E02/RBS01*2-budCkp-Ptrc01/RBS01*2-pflB-Ptrc01/RBS01*2-mhpF-TT07 was derived from pBBR1MCS5-Ptrc01/E02/RBS01*2-budCkp-Ptrc01/RBS01*2-pflB described above and mhpF gene encoding an acetaldehyde dehydrogenase from E. coli.

In this plasmid, expression of mhpF gene was driven by a constitutive trc promoter and optimized RBS sequence. A transcriptional terminator was added at the end of the construct, downstream mhpF gene.

mhpF gene was PCR-amplified with primers Ptrc01-mhpF F (SEQ ID No 13) and Ptrc01-mhpF-TT07 R (SEQ ID No 14) using E. coli MG1655 genomic DNA and cloned into the ApaI and EcoRI restriction sites of the pBBR1MCS5-Ptrc01/E02/RBS01*2-budCkp-Ptrc01/RBS01*2-pflB plasmid (described above). The resulting plasmid was called pBBR1MCS5-Ptrc01/E02/RBS01*2-budCkp-Ptrc01/RBS01*2-pflB-Ptrc01/RBS01*2-mhpF-TT07.

### Construction of Strain 5

The engineered strain which exhibits an increased 2,3-BDO production expressed the pflB gene to produce acetyl-CoA, the mhpF gene to convert acetyl-CoA into acetaldehyde, the PDC1 from Saccharomyces cerevisiae to convert pyruvate and acetaldehyde to acetoin and the synthetic budCkp gene to finally convert acetoin into 2,3-butanediol.

The pCL1920-Ptrc01/E02/RBS01*2-pdc1sc and pBBR1MCS5-Ptrc01/E02/RBS01*2-budCkp-Ptrc01/RBS01*2-pflB-Ptrc01/RBS01*2-mhpF-TT07 were introduced by electroporation into strain 3 (Table 1). The resulting strain MG1655 ΔpoxB ΔldhA ΔackA-pta:: Km (pCL1920-Ptrc01/E02/RBS01*2-pdc1 sc) (pBBR1MCS5-Ptrc01/E02/RBS01*2-budCkp-Ptrc01/RBS01*2-pflB-Ptrc01/RBS01*2-mhpF-TT07) was called strain 5 (Table 1).

### Example 2

### Construction of Strain 7: Overexpression of Acetolactate Synthase, Diacetyl Synthase and Butanediol Dehydrogenase Genes in E. coli

### Construction of Strain 6

In order to keep the pool of pyruvate which is a precursor of the 2,3BDO, the expression of aceEF operon encoding pyruvate dehydrogenase subunit E1 and E2 respectively was attenuated by adding a weak trc constitutive promoter and a transcriptional terminator upstream the aceE gene and by using Ptrc157-aceE F (SEQ ID No 15) and Ptrc157-aceE R (SEQ ID No 16).

Chromosomal attenuation of aceEF operon was introduced into MG1655 using homologous recombinaison (Datsenko & Wanner (2000)) and then transferred by P1 transduction in strain 2 (Table 1). The resulting strain MG1655 ΔpoxB ΔldhA Ptrc157-aceEF::Km was called strain 6 (Table 1).

### Construction of Strain 7

Construction of Plasmid pBBR1MCS5-Ptrc01/E02/RBS01*2-budCkp-Ptrc01/RBS01*2-alsSkpO1ec-TT07

Plasmid pBBR1MCS5-Ptrc01/E02/RBS01*2-budCkp-Ptrc01/RBS01*2-alsSkpO1ec-TT07 was derived from pBBR1MCS5-Ptrc01/E02/RBS01*2-budCkp described in Example 1 and alsSkpO1ec synthetic gene which expression was driven by an independent constitutive trc promoter and an optimized RBS sequence. A transcriptional terminator was added at the end of the construct, downstream alsSkpO1ec gene.

Synthetic Gene alsSkpO1ec

A synthetic gene of the alsS gene from Klebsiella pneumoniae 342 coding for the acetolactate synthase (KPK_{—}2270) was synthesized by Life Technologies (SEQ ID No 55). The construct was cloned into the supplier's pM vector and verified by sequencing:

The alsSkpO1ec synthetic gene was PCR-amplified with primers Ptrc01/RBS01*2-alsSkpO1ec F (SEQ ID No 17) and EcoRI-alsSkpO1ec-TT07 R (SEQ ID No 18) by using the pMK-alsSkpO1ec plasmid provided by the supplier and cloned between the BamHI and EcoRI sites of the pBBR1MCS5-Ptrc01/E02/RBS01*2-budCkp plasmid. The resulting plasmid was called pBBR1MCS5-Ptrc01/E02/RBS01*2-budCkp-Ptrc01/RBS01*2-alsSkpO1ec-TT07.

Construction of Plasmid pCL1920-Ptrc01/E02/RBS01*2-ilv6scO1ec-TT07

Plasmid pCL1920-Ptrc01/E02/RBS01*2-ilv6scO1ec-TT07 was derived from pCL1920 (Lerner & Inouye, 1990, NAR 18, 15 p 4631) and ilv6scO1ec synthetic gene encoding diacetyl synthase from Saccharomyces cerevisiae S288C, which expression was driven by a constitutive trc promoter and optimized RBS sequence. A transcriptional terminator was added at the end of the construct.

Synthetic Gene ilv6scO1ec

Synthetic gene of the ILV6 gene from Saccharomyces cerevisiae S288C coding for the diacetyl synthase small subunit (P25605) was synthesized by Life Technologies (SEQ ID No 56). The construct was cloned into the supplier's pM vector and verified by sequencing. The ilv6scO1ec synthetic gene was PCR-amplified with primers Ptrc01/E02/RBS01*2-ilv6scO1ec F (SEQ ID No 19) and ilv6scO1ec-TT07 R (SEQ ID No 20) using the pMA-ilv6scO1ec plasmid provided by the supplier and cloned between the SacI and KpnI sites of the pCL1920 plasmid. The resulting plasmid was called pCL1920-Ptrc01/E02/RBS01*2-ilv6scO1ec-TT07.

### Construction of Strain 7

The engineered strain which exhibits an increased 2,3-BDO production expressed the optimized alsS gene from Klebsiella pneumoniae to produce acetolactate from pyruvate, the optimized ILV6 gene from Saccharomyces cerevisiae to convert acetolactate into diacetyl and the budC gene from Klebsiella pneumoniae to finally convert diacetyl into acetoin and then into 2,3-butanediol.

Both plasmids pBBR1MCS5-Ptrc01/E02/RBS01*2-budCkp-Ptrc01/RBS01*2-alsSkpO1ec-TT07 and pCL1920-Ptrc01/E02/RBS01*2-ilv6scO1ec-TT07 were introduced by electroporation into strain 6 (Table 1). The resulting strain MG1655 ΔpoxB ΔldhA Ptrc157-aceEF:: Km (pBBR1MCS5-Ptrc01/E02/RBS01*2-budCkp-Ptrc01/RBS01*2-alsSkpO1ec-TT07) (pCL1920-Ptrc01/E02/RBS01*2-ilv6scO1ec-TT07) was called strain 7 (Table 1).

### Example 3

### Construction of Strain 9: Overexpression of Pyruvate Decarboxylase, Pyruvate Formate Lyase, Acetoin Dehydrogenase and Butanediol Dehydrogenase Genes in E. coli

### Construction of Strain 8

In order to increase acetyl-CoA production from pyruvate, pyruvate formate lyase (PFL) NW gene is over-expressed. Since PFL is working as a dimer of PflB, whose maturation requires the enzyme PflAE, the pflA gene was over-expressed. For this purpose, a strong constitutive promoter trc and a RBS consensus sequence was inserted in front of pflA open reading frame at the chromosome by using homologous recombination method described by Datsenko & Wanner (2000).

Overlapping PCR between resistance cassette Cm fragment (Ptrc01/E06/RBS01*2-pflA F (SEQ ID No 21) and Ptrc01-pflA R (SEQ ID No 22) and fragment homologous to the beginning of pflA gene (pflA R (SEQ ID No 23) and pflA F (SEQ ID No 24) was done by using pflA F (SEQ ID No 24) and RI pflB R (SEQ ID No 25) was performed and introduced in MG1655 strain. Chromosomal modification of pflA promoter was then transferred by P1 transduction in strain 3 (Table 1). The resulting strain MG1655 ΔpoxB ΔldhA ΔackA-pta::Km Ptrc01/E06/RBS01*2-pflA::Cm was called strain 8 (Table 1)

### Construction of Strain 9

Construction of Plasmid pBBR1MCS5-Ptrc01/E02/RBS01*2-budCkp-Ptrc01/RBS01*2-pflB-UTR-acoABbs-TT07

The plasmid pBBR1MCS5-Ptrc01/E02/RBS01*2-budCkp-Ptrc01/RBS01*2-pflB-UTR-acoABbs-TT07 was derived from the pBBR1MCS5-Ptrc01/E02/RBS01*2-budCkp-Ptrc01/RBS01*2-pflB plasmid described in Example 1 and acoA/acoB operon from Bacillus subtilis 168 encoding the acetoin dehydrogenase subunits, alpha and beta respectively which expression was driven by the same constitutive Ptrc promoter than pflB gene and their own 5′ untranslated region. A transcriptional terminator was added at the end of the construct.

Construction of pBBR1MCS5-Ptrc01/E02/RBS01*2-budCkp-Ptrc01/RBS01*2-pflB-UTR-acoABbs-TT07 Plasmid

The UTR-acoABbs-TT07 fragment was PCR-amplified with primers Ptrc01/UTR-acoAB IF F (2) (SEQ ID No 26) and acoABbs-TT07 IF R (SEQ ID No 27) using Bacillus subtilis 168 genomic DNA and cloned into the HindIII and EcoRI restriction sites of the pBBR1MCS5-Ptrc01/E02/RBS01*2-budCkp-Ptrc01/RBS01*2-pflB plasmid (described in Example 1). The resulting plasmid was called pBBR1MCS5-Ptrc01/E02/RBS01*2-budCkp-Ptrc01/RBS01*2-pflB-UTR-acoABbs-TT07.

### Construction of Strain 9

The engineered strain which exhibits an increased 2,3-BDO production expressed the pflB and pflA genes to produce acetyl-CoA, the PDC1 from Saccharomyces cerevisiae to convert pyruvate into acetaldehyde, the natural acoA and acoB genes from Bacillus subtilis to condense acetyl-CoA and acetaldehyde into acetoin and the optimized budC gene from Klebsiella pneumoniae to finally convert acetoin into 2,3-butanediol.

Plasmids pCL1920-Ptrc01/E02/RBS01*2-pdc1sc and pBBR1MCS5-Ptrc01/E02/RBS01*2-budCkp-Ptrc01/RBS01*2-pflB-UTR-acoABbs-TT07 were both introduced by electroporation into strain 8 (Table 1). The resulting strain MG1655 ΔpoxB ΔldhA ΔackA-pta::Km Ptrc01/E06/RBS01*2-pflA:: Cm (pCL1920-Ptrc01/E02/RBS01*2-pdc1sc) (pBBR1MCS5-Ptrc01/E02/RBS01*2-budCkp-Ptrc01/RBS01*2-pflB-UTR-acoABbs-TT07) was called strain 9 (Table 1).

### Example 4

### Construction of Strain 13: Overexpression of Pyruvate Formate Lyase, Pyruvate Formate Lyase Activating Enzyme, Acetaldehyde Dehydrogenase and Butanediol Dehydrogenase Genes in S. cerevisiae

### Construction of Strain 11

In order to prevent ethanol production, deletions of genes ADH1, ADH3 and ADH5 encoding the three major alcohol dehydrogenases were constructed successively by employing the lox::kanMX::loxP/Cre recombinase system and the short flanking homology PCR technology as described by Guldener et al. (1996).

Each gene was deleted by introducing a gene deletion cassette containing loxP-kanMX-loxP conferring geneticin resistance and homologous region upstream and downstream the coding region of the gene of interest. Each deletion cassette was PCR-amplified from plasmid pUG6 (Guldener et al., 1996) with primers adh1::kanMXlox fwd (SEQ ID No 28) and adh1::kanMXloxrev (SEQ ID No 29) for ADH1 gene deletion, adh3::kanMXlox fwd (SEQ ID No 30) and adh3::kanMXloxrev (SEQ ID No 31) for ADH3 gene deletion and Adh5::kanMXlox fwd (SEQ ID No 32) and Adh5::KanMXloxrev (SEQ ID No 33) for ADH5 gene deletion. PCR products were transformed into yeast as described by Gietz and al. (2002 and 2007). The loxP-kanMX-loxP selection cassette at ADH1 locus and then at ADH3 locus was removed via transformation with pSH47 encoding galactose-inducible Cre recombinase (Guldener et al., 1996). The selected and verified strain CEN.PK2-1C Δadh1 Δadh3 Δadh5::Gn was called strain 11. (Table 1).

### Construction of Strain 12

In order to eliminate the 2.3BDO natural production pathway in yeast, ILV2 gene deletion which encodes the catalytic subunit of the acetolactate synthase has been carried out as described previously, except that, instead of geneticin resistance cassette, hygromycine B resistance (hphNT1) resistance cassette was amplified by using ilv2::hphNT1loxrfwd (SEQ ID No 34) and ilv2::hphNT1loxrev (SEQ ID No 35) primers. PCR product was transformed into strain 11 (Table 1) as described by Gietz et al. (2002 and 2007). The selected and verified strain CEN.PK2-1C Δadh1 Δadh3 Δadh5::Gn Δilv2::Hg was called strain 12 (Table 1).

### Construction of Strain 13

Construction of Plasmid p424-Phxt7-bdh1-TTcyc1

The plasmid p424-Phxt7-bdh1-TTcyc1 was derived from p424H7 plasmid (Wieczorke et al. 1999 and Hamacher et al. 2002) and BDH1 gene encoding the most active 2,3-butanediol dehydrogenase under the control of truncated HXT7 promoter and CYC1 terminator was added at the end of the gene for transcription termination.

Coding region of BDH1 was PCR-amplified with A-BDH1-f (SEQ ID No 36) and A-BDH1-r (SEQ ID No 37) from S. cerevisiae S288C genomic DNA and cloned by recombination cloning into the p424H7 plasmid digested by BamHI/XhoI. The verified and sequenced plasmid was called p424-Phxt7-bdh1-TTcyc1.

Construction of Plasmid p426-Phxt7-mhpFec-TTcyc1

The plasmid p426-Phxt7-mhpFec-TTcyc1 was derived from p426H7 plasmid (Wieczorke et al. 1999 and Hamacher et al. 2002) and mhpF gene encoding the acetaldehyde dehydrogenase from Escherichia coli under the control of truncated HXT7 promoter and the CYC1 terminator was added at the end of the gene for transcription termination.

Coding region of mhpF gene was PCR-amplified with mhpF-f (SEQ ID No 38) and mhpF-r (SEQ ID No 39) from E. coli MG1655 genomic DNA and cloned by recombination cloning into the p426H7 plasmid digested by BamHI/XhoI. The verified and sequenced plasmid was called p426-Phxt7-mhpFec-TTcyc1.

Construction of Plasmid p423-Phxt7-pflAec-TTcyc1-Ppgk1-pflBec-TTpdc1

The plasmid p423-Phxt7-pflAec-TTcyc1-Ppgk1-pflBec-TTpdc1 was derived from p423H7 plasmid (Wieczorke et al. 1999 and Hamacher et al. 2002). The gene pflA gene encoding the pyruvate formate lyase activating enzyme from Escherichia coli was cloned under the control of truncated HXT7 promoter and the pflB gene encoding the pyruvate formate lyase cloned under the control of PGK1 promoter. The CYC1 and PDC1 terminators were respectively added at the end of the pflA and pflB genes for transcription termination.

Coding regions of pflA and pflB genes were PCR-amplified with the following primers:
pflA-f (SEQ ID No 40) and pflA-r (SEQ ID No 41);
A-EcPFLB-f (SEQ ID No 42) and A-EcPFLB-r (SEQ ID No 43), respectively by using E. coli MG1655 genomic DNA.

CYC1 and PDC1 terminators and PGK1 promoter were amplified with the following primers:
A-tCYC1-f (SEQ ID No 44) and A-tCYC1-r (SEQ ID No 45)
A-tPDC1-f (SEQ ID No 46) and A-tPDC1-r (SEQ ID No 47)
A-pPGK1-f (SEQ ID No 48) and A-pPGK1-r (SEQ ID No 49), respectively by using S. cerevisiae S288C genomic DNA.

Assembly of each PCR fragment into the p423H7 plasmid digested by BamHI/PsiI was done by recombination cloning. The verified and sequenced plasmid was called p423-Phxt7-pflAec-TTcyc1-Ppgk1-pflBec-TTpdc1.

### Construction of Strain 13

The engineered strain which exhibits an increased 2,3-BDO production expressed the pflB and pflA genes from E. coli to produce acetyl-CoA, the mhpF gene from E. coli to convert acetyl-CoA into acetaldehyde, the endogenous expression of PDC1 gene to convert pyruvate and acetaldehyde into acetoin and the overexpressed BDH1 gene to finally convert acetoin into 2,3-butanediol.

Plasmids p424-Phxt7-bdh1-TTcyc1, p426-Phxt7-mhpFec-TTcyc1 and p423-Phxt7-pflAec-TTcyc1-Ppgk1-pflBec-TTpdc1 were introduced into strain 12 (Table 1) by transformation according to Gietz et al., (2007) protocol. The resulting strain CEN.PK2-1C Δadh1 Δadh3 Δadh5::Gn Dilv2::Hg (p424-Phxt7-bdh1-TTcyc1) (p426-Phxt7-mhpFec-TTcyc1)(p423-Phxt7-pflAec-TTcyc1-Ppgk1-pflBec-TTpdc1) was called strain 13

### Example 5

### Construction of Strain 14: Overexpression of Pyruvate Formate Lyase, Pyruvate Formate Lyase Activating Enzyme, Acetoin Reductase and Butanediol Dehydrogenase Genes in S. cerevisiae

### Construction of Strain 14

Construction of Plasmid p426-Phxt7-acoAbs-TTcyc1-Ppgk1-acoBbs-TTpdc1

In order to improve the condensation of acetyl-CoA and acetaldehyde into acetoin, the acetoin reduction complex acoAB from Bacillus subtilis was overexpressed.

The plasmid p426-Phxt7-acoAbs-TTcyc1-Ppgk1-acoBbs-TTpdc1 was derived from p426H7 plasmid (Wieczorke et al. 1999 and Hamacher et al. 2002) and acoAbs/acoBbs genes under the control of truncated HXT7 and PGK1 promoters respectively. The CYC1 and PDC1 terminators were added, respectively, at the end of the acoA and acoB gene for transcription termination.

Coding region of acoA and acoB genes were PCR-amplified with the following primers:
A-acoA-f (SEQ ID No 50) and A-acoA-r (SEQ ID No 51)
A-acoB-f (SEQ ID No 52) and acoB-r (SEQ ID No 53) respectively by using Bacillus subtilis 168 genomic DNA.

CYC1 and PDC1 terminators and PGK1 promoter were amplified as described in example 4.

Assembly of each PCR fragment into the p426H7 plasmid digested by BamHI/KpnI was done by recombination cloning. The verified and sequenced plasmid was called p426-Phxt7-acoAbs-TTcyc1-Ppgk1-acoBbs-TTpdc1.

### Construction of Strain 14

The engineered strain which exhibits an increased 2,3-BDO production expressed the pflB and pflA genes to produce acetyl-CoA, the endogenous expression of PDC1 to convert pyruvate to acetoin, the acoA and acoB genes from Bacillus subtilis to condense acetyl-CoA and acetaldehyde into acetoin and the overexpressed BDH1 gene to finally convert acetoin into 2,3-butanediol.

Plasmids p424-Phxt7-bdh1-TTcyc1, p426-Phxt7-acoAbs-TTcyc1-Ppgk1-acoBbs-TTpdc1 and p423-Phxt7-pflAec-TTcyc1-Ppgk1-pflBec-TTpdc1 were introduced into strain 12 (Table 1) by transformation according to Gietz et al. (2007) protocol. The resulting strain CEN.PK2-1C Δadh1 Δadh3 Δadh5::Gn Dilv2::Hg (p424-Phxt7-bdh1-TTcyc1) (p426-Phxt7-acoAbs-TTcyc1-Ppgk1-acoBbs-TTpdc1) (p423-Phxt7-pflAec-TTcyc1-Ppgk1-pflBec-TTpdc1) was called strain 14 (Table 1).

### Example 6

### Production of 2.3BDO in Shake Flask

E. coli

Pathway 3

Production strains were evaluated in small Erlenmeyer flasks using modified M9 medium (Anderson, 1946) that is supplemented with 5 g·L⁻¹ of yeast extract, 10 g·L⁻¹ MOPS and 10 g·L⁻¹ glucose and adjusted to pH 6.8.

A 25 mL preculture was grown at 37° C. for 15 hours in a mixed medium (10% LB medium (Sigma 25%) with 2.5 g·L⁻¹ glucose and 90% minimal medium described above). It was used to inoculate a 50 mL culture to an OD₆₀₀ of 0.3 in modified minimal medium.

When necessary, antibiotics were added at concentrations of 50 mg·L⁻¹ for kanamycin and spectinomycin and 10 mg·L⁻¹ for gentamycin. The temperature of the cultures is 37° C. and the agitation is set up at 200 RPM. When the culture reached an OD₆₀₀ of 5 to 6, extracellular metabolites were analyzed using HPLC with refractometric detection (organic acids and glucose). Production of BDO was determined by LCMSMS.

TABLE 2
BDO titer for strains 1 and 7 with aerobic conditions.
For each strain, two repetitions were made.

| Strain | [BDO] (mg · L⁻¹) |
|---|---|
| 1 | Not detected |
| 7 | 41 |

As can be seen in table 2, BDO production is increased upon overexpression of ILV6, budC and alsS.

Pathways 2 and 4

Production strains were evaluated in small flasks using modified MAC medium that is supplemented with 10 g·L⁻¹ glucose and adjusted to pH 6.8.

A 25 mL preculture was grown at 37° C. for 15 hours in a mixed medium (10% LB medium (Sigma 25%) with 2.5 g·L⁻¹ glucose and 90% M9 medium (Anderson, 1946). It was used to inoculate a 50 mL culture to an OD₆₀₀ of 0.3 in MAC medium. The composition of this medium was in g per liter: glycerol: 20.0; tryptone: 10.0; NaCl: 5.0; NaNO₃ 0.085; yeast extract: 5.0; K₂HPO₄: 0.5; FeSO₄.7H₂O: 0.05; HEPES: 23.0 and NTA: 0.2.

When necessary, antibiotics were added at concentrations of 50 mg·L⁻¹ for kanamycin and spectinomycin, of 30 mg·L⁻¹ for chloramphenicol and 10 mg·L⁻¹ for gentamycin. The temperature of the cultures is 37° C., and the agitation set up 200 RPM. Microaerobic conditions were applied to the culture by sealing flasks with rubber stoppers. After 7 hours, extracellular metabolites were analyzed using HPLC with refractometric detection (organic acids and glucose). Production of BDO was determined by LCMSMS. Each strain was evaluated twice.

TABLE 3
BDO titer for strains 1, 4 and 9 with microaerobic
conditions for pathway 4

| Strain | [BDO] (mg · L⁻¹) |
|---|---|
| 1 | 0 |
| 9 | 118 |
| 4 | 111 |

TABLE 4
BDO titer for strains 1, 4 and 5 with microaerobic conditions
for pathway 2. For each strain, two repetitions were made.

| Strain | [BDO] (mg · L⁻¹) |
|---|---|
| 1 | 0 |
| 5 | 257 |
| 4 | 211 |

BDO production is increased with overexpression of acetolactate synthase, diacetyl synthase and butanediol dehydrogenase genes in E. coli.

S. cerevisiae Yeast

Production strains were assessed in small flasks. A 7.5 mL preculture was grown at 30° C. for 17 hours in YPD medium (10 g·L⁻¹ yeast extract, 10 g·L⁻¹ Bactopeptone and 10 g·L⁻¹ glucose). It was used to inoculate a 70 mL culture of MM1 medium to reach an OD₆₀₀ of 0.2. Synthetic mineral medium (MM1) was prepared as follows (all concentrations in g·L⁻¹): glucose: 10.0; (NH₄)₂SO₄: 11.0; KH₂PO₄: 5.5; MgSO₄.7H₂O: 0.9; EDTA.2Na.2H₂O: 0.0300; ZnSO₄.7H₂O: 0.0090; CoCl₂ 6H₂O: 0.0006; MnCl₂.4H₂O: 0.0020; CuSO₄.5H₂O: 0.0006; FeSO₄.7H₂O: 0.0060; Na₂MoSO₄.2H2O: 0.0080; H₃BO₃: 0.0020; CaCl₂.2H₂O: 0.0090; biotin: 0.0020; calcium pantothenate: 0.0020; nicotinic acid: 0.0020; myoinositol: 0.0050; hydrochloride thiamine: 0.0020; para-aminobenzoic acid: 0.0004 and pyridoxine: 0.0020.

When necessary, leucine is added at a final concentration of 0.44 mM in the medium.

The temperature of the culture was maintained at 30° C. and with an agitation of 200 RPM. Yeasts were cultivated in aerobic, microaerobic and anaerobic conditions.

Microaerobic conditions were applied to the culture by sealing flasks with rubber stoppers. For anaerobic conditions, 70 mL of minimal media were supplemented with 0.42 g·L⁻¹ Tween 80 and 0.04 g·L⁻¹ cholesterol. Bottles were purge with pure nitrogen gas for 15 minutes to establish anaerobic conditions. After about 45 hours, BDO was quantified by LCMSMS and other relevant metabolites were analyzed using HPLC with refractometric detection (organic acids and glucose).

With strains 13 and 14, BDO production was effective for all the evaluated conditions. Final concentrations were comprised between 80 and 130 mg·L⁻¹ for anaerobic conditions and between 187 and 234 mg·L⁻¹ for aerobic conditions. Performances obtained in microaerobic conditions are presented in table 5.

TABLE 5
BDO and ethanol titers for strains 10, 13 and 14 in microaerobic
conditions. For each strain, two repetitions were made.

| | BDO | Ethanol |
|---|---|---|
| Strain | (mg · L⁻¹) | (g · L⁻¹) |
| 10 | 6 | 3.9 |
| 13 | 307 | 0.3 |
| 14 | 341 | 0.5 |

As can be seen in table 5, BDO production is increased upon overexpression of the two pathways introduced in S. cerevisiae.

### REFERENCES

### Patent References

WO2011041426
WO2010151525
WO2004076659

### Non-Patent References

Altschul S F (1993), J Mol Evol. 36(3):290-300.
Anderson E H (1946), Proc. Natl. Acad. Sci. USA 32:120-128.
Bassit N, Boquien C Y, Picque D, Corrieu G. (1993), Appl Environ Microbiol. 59(6):1893-7.
Baudin A, Ozier-Kalogeropoulos O, Denouel A, Lacroute F, Cullin C. (1993), Nucleic Acids Res. 21(14):3329-3330
Brat D, Weber C, Lorenzen W, Bode H B, Boles E. (2012), Biotechnology for Biofuels. 5:65-80.
Boles E, Hollenberg C P. (1997) FEMS Microbiol Rev. 21(1):85-111.
Celińska E, Grajek W. (2009), Biotechnol Adv. 27(6):715-25.
Danner H, Braun R. (1999), Chem. Soc. Rev., 28:395-405.
Datsenko K A, Wanner B L (2000), Proc Natl Acad Sci USA. 97: 6640-6645.
Ehsani M, Fernández M R, Biosca J A, Julien A, Dequin S. (2009), Appl Environ Microbiol. 75(10):3196-205.
Garg S K, Jain A. (1995), Bioresour Technol. 51:103-109.
Gietz R D, Woods R A. (2002), Methods Enzymol. 350:87-96.
Gietz R D, Schiestl R H. (2007), Nat. Protoc. 2:31-34.
González E, Fernandez M R, Marco D, Calam E, Sumoy L, Parés X, Dequin S, Biosca J A. (2010), Appl Environ Microbiol. 76(3):670-9.
Güldener U, Heck S, Fielder T, Beinhauer J, Hegemann J H. (1996), Nucleic Acids Res. 24(13):2519-24.
Gueldener U, Heinisch J, Koehler G J, Voss D, Hegemann J H. (2002), Nucleic Acids Res. 30(6):e23.
Guthrie & Fink, (2004) Methods in Enzymology, Volume 194, Guide to Yeast Genetics and Molecular and Cell Biology (Part A, 2004, Christine Guthrie and Gerald R. Fink (Eds), Elsevier Academic Press, San Diego, Calif.
Hahn-Hägerdal B, Karhumaa K, Jeppsson M, Gorwa-Grauslund M F. (2007), Adv Biochem Eng Biotechnol. 108:147-77.
Hamacher T, Becker J, Gárdonyi M, Hahn-Hagerdal B, Boles E. (2002), Microbiology. 148(Pt 9):2783-8.
Hasty et al. (1991), Mol Cell Biol 11:5586-91.
Hatti-Kaul R, Törnvall U, Gustafsson L, Börjesson P. (2007), Trends Biotechnol. 25(3):119-24.
Jin Y S, Jeffries T W. (2004), Metab Eng. 6(3):229-38.
Jin Y S, Laplaza J M, Jeffries T W. (2004), Appl Environ Microbiol. 70(11):6816-25.
Ji X J, Huang H, Ouyang P K. (2011), Biotechnol Adv. 29(3):351-64.
Larsson C, Nilsson A, Blomberg A, Gustafsson L. (1997), J Bacteriol. 179(23):7243-50.
Larsson C, Påhlman I L, Gustafsson L. (2000), Yeast. 16(9):797-809.
Liu M, Sergienko E A, GuoF, Wang J, Tittmann K, Hubner G, Furey W, Jordan F. (2011), Biochemistry. 40:7355-7368.
Ma H, Kunes S, Schatz P J, Botstein D. (1987), Gene. 58:201-2016.
Magee R J, Kosaric N. (1985), Adv Biochem Eng Biotechnol. 32:61-93.
Miller (1992), A Short Course in Bacterial Genetics: A Laboratory Manual and Handbook for Escherichia coli and Related Bacteria, Cold Spring Harbor Laboratory Press, Cold Spring Harbor, N.Y.
Nevoigt E, Fischer C, Mucha O, Matthäus F, Stahl U, Stephanopoulos G. (2007), Biotechnol Bioeng. 96(3):550-8.
Ng C Y, Jung M, Lee J, Oh M. (2012), Microbial Cell Factories. 11: 68-81.
Nielsen D R, Yoon S H, Yuan C J, Prather K L. (2010), Biotechnol J. 5(3):274-84.
Portnoy V A, Scott D A, Lewis N E, Tarasova Y, Osterman A L, Palsson B O. (2010), Appl Environ Microbiol. 76(19):6529-40.
Raymond C K, Pownder T A, Sexson S. (1999), BioTechniques. 26:134-141.
Romano P, Suzzi G. (1996), Appl Environ Microbiol. 62(2):309-15.
Romano P, Suzzi G, Brandolini V, Menziani E, Domizio P. (1996), Lett Appl Microbiol. 22(4):299-302.
Runquist D, Hahn-Hagerdal B, Rådström P. (2010), Biotechnol Biofuels. 3:5.
Saloheimo A, Rauta J, Stasyk O V, Sibirny A A, Penttilä M, Ruohonen L. (2007), Appl Microbiol Biotechnol. 74(5):1041-52.
Sambrook J et al. (1989) (2001), “Molecular Cloning: A Laboratory Manual” 2nd & 3rd Editions, Cold Spring Harbor Laboratory Press.
Schaefer U, Boos W, Takors R, Weuster-Botz D., (1999), Anal. Biochem. 270: 88-96.
Sedlak M, Ho N W. (2004), Yeast. 21(8):671-84.
Smidt O, Preez J C, Albertyn J. (2012), FEMS Yeast Res. 12(1):33-47.
Sunderland, Mass., or Deshpande, Mukund V. (1992), Appl. Biochem. Biotechnol., 36:227.
Syu M J. (2001), Appl Microbiol Biotechnol. 55(1):10-8.
Takasaki K, Shoun H, Yamaguchi M, Takeo K, Nakamura A, Hoshino T, Takaya N. (2004), J Biol Chem. 279(13):12414-20.
Ui S, Takusagawa Y, Sato T, Ohtsuki T, Mimura A, Ohkuma M, Kudo T. (2004), Lett Appl Microbiol. 39(6):533-7.
Wach A, Brachat A, Pohlmann R, Philippsen P. (1994), Yeast. 10:1793-1808.
Wieczorke R, Krampe S, Weierstall T, Freidel K, Hollenberg C P, Boles E. (1999), FEBS Lett. 464(3):123-8.
Wu K J, Saratale G D, Lo Y C, Chen W M, Tseng Z J, Chang M C, Tsai B C, Su A, Chang J S. (2008), Bioresour Technol. 99(17):7966-70.
Zhou Z, Takaya N, Nakamura A, Yamaguchi M, Takeo K, Shoun H. (2002), J Biol Chem. 277(3):1892-6.

1. A recombinant microorganism engineered for production of 2,3-butanediol (BDO) wherein said microorganism overexpresses at least one gene encoding a polypeptide involved in conversion of pyruvate into 2,3-butanediol.

2. The recombinant microorganism of claim 1 wherein conversion of pyruvate into 2,3-butanediol comprises:
a) conversion of pyruvate to acetaldehyde or to acetyl-CoA and then conversion of acetyl-CoA to acetaldehyde,
b) conversion of pyruvate and/or acetaldehyde to acetoin and
c) conversion of acetoin to 2,3-butanediol.

3. The recombinant microorganism of claim 2 wherein said microorganism expresses the following enzymes:
a) a polypeptide catalysing conversion of pyruvate to acetaldehyde or two polypeptides, one catalysing conversion of pyruvate to acetyl-CoA and another catalysing conversion of acetyl-CoA to acetaldehyde, and
b) a polypeptide catalysing condensation of pyruvate and acetaldehyde to acetoin or a polypeptide catalysing condensation of acetaldehyde to acetoin, and
c) a butanediol dehydrogenase.

4. The recombinant microorganism of claim 3 wherein the microorganism expresses one gene selected from the group consisting of PDC1, PDC5 and PDC6 encoding pyruvate decarboxylase, pflA and/or pflB genes encoding pyruvate formate lyase and mhpF gene encoding acetaldehyde dehydrogenase.

5. The recombinant microorganism of claim 1 wherein conversion of pyruvate into 2,3-butanediol comprises:
a) conversion of pyruvate to acetaldehyde and to acetyl-CoA,
b) condensation of acetaldehyde and acetyl-CoA to acetoin,
c) conversion of acetoin into 2,3-butanediol.

6. The recombinant microorganism of claim 5 wherein said microorganism expresses the following enzymes:
a) a polypeptide catalysing conversion of pyruvate to acetaldehyde and a polypeptide catalysing conversion of pyruvate to acetyl-CoA, and
b) a polypeptide catalysing condensation of acetyl-CoA and acetaldehyde to acetoin, and
c) a butanediol dehydrogenase (BDH).

7. The recombinant microorganism of claim 6 wherein the microorganism expresses:
one gene selected from the group consisting of PDC1, PDC5 and PDC6 encoding pyruvate decarboxylase and pflA and/or pflB genes encoding pyruvate formate lyase and
acoA and/or acoB genes encoding acetoin dehydrogenase.

8. The recombinant microorganism of claim 1 wherein conversion of pyruvate into 2,3-butanediol comprises:
a) conversion of pyruvate to acetolactate,
b) conversion of acetolactate to diacetyl,
c) conversion of diacetyl to acetoin,
d) conversion of acetoin to 2,3-butanediol.

9. The recombinant microorganism of claim 1 wherein conversion of pyruvate into 2,3-butanediol comprises:
a) conversion of pyruvate to acetolactate,
b) conversion of acetolactate to acetoin,
c) conversion of acetoin to 2,3-butanediol.

10. The recombinant microorganism of claim 9, wherein the microorganism expresses genes encoding polypeptides having activity of acetolactate synthase (ALS) and butanediol dehydrogenase (BDH).

11. The recombinant microorganism according to claim 9 wherein the microorganism expresses gene encoding polypeptide having activity of acetolactate synthase (ALS) encoded by the gene budB from Klebsiella pneumoniae or the gene ilv2 from Saccharomyces cerevisiae and gene encoding polypeptide having activity of butanediol dehydrogenase (BDH).

12. The recombinant microorganism of claim 8 wherein diacetyl is converted into 2,3-butanediol by BDH.

13. The recombinant microorganism of claim 9 wherein said microorganism expresses acetolactate decarboxylase (ALDC).

14. The recombinant microorganism of claim 13 wherein the polypeptide having an ALDC activity is encoded by the gene budA from Klebsiella pneumoniae.

15. The recombinant microorganism according to claim 9, wherein expression of at least one gene selected from the group consisting of PDC1, PDC5 and PDC6 is attenuated.

16. The recombinant microorganism according to claim 6, wherein BDH is encoded by budC from Klebsiella pneumoniae, BDH1 from Saccharomyces cerevisiae, or BDH2 from Saccharomyces cerevisiae.

17. The recombinant microorganism of claim 1, wherein the microorganism is further modified to overexpress at least one gene encoding for: aldose reductase, xylitol dehydrogenase, xylulokinase, L-arabinol-4-dehydrogenase, L-xylulose reductase, hexose transporter and/or galactose transporter.

18. The recombinant microorganism of claim 1, wherein said microorganism is selected from the group consisting of Saccharomycetaceae species and Enterobacteriaceae species.

19. A method of production of 2,3-butanediol (BDO) comprising:
providing a recombinant microorganism according to claim 1,
cultivating the recombinant microorganism in a culture medium comprising a source of carbon, and
recovering the 2,3-butanediol.


SEQUENCE LISTING (56 sequences)

SEQ ID NO: 1 - DNA, 99 bp, Artificial Sequence
  Oligonucleotide
ccttagccagtttgttttcgccagttcgatcacttcatcaccgcgtccgctgatgattgcgcgcagcatatacaggctgcatatgaatatcctccttag

SEQ ID NO: 2 - DNA, 102 bp, Artificial Sequence
  Oligonucleotide
cggttgcagcttatatcgccaaaacactcgaatcggcaggggtgaaacgcatctggggagtcacaggcgactctctgaacggtgtaggctggagctgcttcg

SEQ ID NO: 3 - DNA, 101 bp, Artificial Sequence
  Oligonucleotide
ttaaaccagttcgttcgggcaggtttcgcctttttccagattgcttaagttttgcagcgtagtctgagaaatactggtcagcatatgaatatcctccttag

SEQ ID NO: 4 - DNA, 100 bp, Artificial Sequence
  Oligonucleotide
gaaactcgccgtttatagcacaaaacagtacgacaagaagtacctgcaacaggtgaacgagtcctttggctttgagctggtgtaggctggagctgcttcg

SEQ ID NO: 5 - DNA, 100 bp, Artificial Sequence
  Oligonucleotide
cgagtaagttagtactggttctgaactgcggtagttcttcactgaaatttgccatcatcgatgcagtaaatggtgaagagtgtaggctggagctgcttcg

SEQ ID NO: 6 - DNA, 97 bp, Artificial Sequence
  Oligonucleotide
gctgctgtgcagactgaatcgcagtcagcgcgatggtgtagacgatatcgtcaaccagtgcgccacgggacaggtcgcatatgaatatcctccttag

SEQ ID NO: 7 - DNA, 104 bp, Artificial Sequence
  Oligonucleotide
ccggagctcgagctgttgacaattaatcatccggctcgtataatgtgtggaagtcgacgttaaccctaggtaaggaggttataaatgtctgaaattactttggg

SEQ ID NO: 8 - DNA, 38 bp, Artificial Sequence
  Oligonucleotide
cgcggatccgcggctagcttattgcttagcgttggtag

SEQ ID NO: 9 - DNA, 107 bp, Artificial Sequence
  Oligonucleotide
cgggagctcgagctgttgacaattaatcatccggctcgtataatgtgtggaagtcgacgttaaccctaggtaaggaggttataaatgaaaaaagtcgcacttgttac

SEQ ID NO: 10 - DNA, 28 bp, Artificial Sequence
  Oligonucleotide
gctctagattagttaaacaccatcccgc

SEQ ID NO: 11 - DNA, 86 bp, Artificial Sequence
  Oligonucleotide
ggactagtgagctgttgacaattaatcatccggctcgtataatgtgtggaataaggaggttataaatgtccgagcttaatgaaaag

SEQ ID NO: 12 - DNA, 28 bp, Artificial Sequence
  Oligonucleotide
cggaattcttacatagattgagtgaagg

SEQ ID NO: 13 - DNA, 100 bp, Artificial Sequence
  Oligonucleotide
atctatgtaagaattcgagctgttgacaattaatcatccggctcgtataatgtgtggaataaggaggttataaatgagtaagcgtaaagtcgccattatc

SEQ ID NO: 14 - DNA, 68 bp, Artificial Sequence
  Oligonucleotide
aaaagctgggtaccgggcccgcagaaaggcccacccgaaggtgagccagtcatgccgcttctcctgcc

SEQ ID NO: 15 - DNA, 103 bp, Artificial Sequence
  Oligonucleotide
ctaaacgtagaacctgtcttattgagctttccggcgagagttcaatgggtcacactggctcaccttcgggtgggcctttctgccatatgaatatcctccttag

SEQ ID NO: 16 - DNA, 121 bp, Artificial Sequence
  Oligonucleotide
cgttctgacatgggttattccttatctatctaataacgttgagttttctggaacctgtttccacacagtatacgagccggatgattaatcgacaacagctctgtaggctggagctgcttcg

SEQ ID NO: 17 - DNA, 85 bp, Artificial Sequence
  Oligonucleotide
cgggatccgagctgttgacaattaatcatccggctcgtataatgtgtggaataaggaggttataaatggataaacagtatccggt

SEQ ID NO: 18 - DNA, 57 bp, Artificial Sequence
  Oligonucleotide
cggaattcgcagaaaggcccacccgaaggtgagccagttacagaatctgtgacagat

SEQ ID NO: 19 - DNA, 108 bp, Artificial Sequence
  Oligonucleotide
cgggagctcgagctgttgacaattaatcatccggctcgtataatgtgtggaagtcgacgttaaccctaggtaaggaggttataaatgctgcgtagcctgctgcaaagc

SEQ ID NO: 20 - DNA, 58 bp, Artificial Sequence
  Oligonucleotide
gcggtaccgcagaaaggcccacccgaaggtgagccagttaacccggaggcagctggct

SEQ ID NO: 21 - DNA, 111 bp, Artificial Sequence
  Oligonucleotide
gaatgcgaccaataactgacatttataacctccttaggatccgtcgacttccacacattatacgagccggatgattaattgtcaacagctccatatgaatatcctccttag

SEQ ID NO: 22 - DNA, 120 bp, Artificial Sequence
  Oligonucleotide
ctatctatactttaaggtgactgccaaaacagactcgacgtagccttcgagctgcgcaccaacacggcctcagatgggccacatctggagaaacaccgcatgtaggctggagctgcttcg

SEQ ID NO: 23 - DNA, 20 bp, Artificial Sequence
  Oligonucleotide
gtcagttattggtcgcattc

SEQ ID NO: 24 - DNA, 19 bp, Artificial Sequence
  Oligonucleotide
ctcgtcgttcatctgtttg

SEQ ID NO: 25 - DNA, 26 bp, Artificial Sequence
  Oligonucleotide
gctatctatactttaaggtgactgcc

SEQ ID NO: 26 - DNA, 115 bp, Artificial Sequence
  Oligonucleotide
atctatgtaagaattcgctagcgagctgttgacaattaatcatccggctcgtataatgtgtggaagaaccctaaatagaaggaggcgcacaaaatgaaattgttaaaacgagaag

SEQ ID NO: 27 - DNA, 71 bp, Artificial Sequence
  Oligonucleotide
cggtatcgataagcttgcagaaaggcccacccgaaggtgagccagtacgtattaattcaatgccggctcgc

SEQ ID NO: 28 - DNA, 62 bp, Artificial Sequence
  Oligonucleotide
tcaagctataccaagcatacaatcaactatctcatatacagcataggccactagtggatctg

SEQ ID NO: 29 - DNA, 60 bp, Artificial Sequence
  Oligonucleotide
cttatttaataataaaaatcataaatcataagaaattcgcccagctgaagcttcgtacgc

SEQ ID NO: 30 - DNA, 62 bp, Artificial Sequence
  Oligonucleotide
gttaaaactaggaatagtatagtcataagttaacaccatcgcataggccactagtggatctg

SEQ ID NO: 31 - DNA, 60 bp, Artificial Sequence
  Oligonucleotide
acaaagactttcataaaaagtttgggtgcgtaacacgctaccagctgaagcttcgtacgc

SEQ ID NO: 32 - DNA, 62 bp, Artificial Sequence
  Oligonucleotide
aagaaaattatttaactacatatctacaaaatcaaagcatgcataggccactagtggatctg

SEQ ID NO: 33 - DNA, 60 bp, Artificial Sequence
  Oligonucleotide
taaaaagtaaaaatatattcatcaaattcgttacaaaagaccagctgaagcttcgtacgc

SEQ ID NO: 34 - DNA, 70 bp, Artificial Sequence
  Oligonucleotide
ctaaaccctttgagctaagaggagataaatacaacagaatcaattttcaattcgtacgctgcaggtcgac

SEQ ID NO: 35 - DNA, 70 bp, Artificial Sequence
  Oligonucleotide
gtctgcattttttactgaaaatgcttttgaaataaatgtttttgaaatgcataggccactagtggatctg

SEQ ID NO: 36 - DNA, 65 bp, Artificial Sequence
  Oligonucleotide
gaataaacacaaaaacaaaaagtttttttaattttaatcaaaaaatgagagctttggcatatttc

SEQ ID NO: 37 - DNA, 67 bp, Artificial Sequence
  Oligonucleotide
cggatgtggggggagggcgtgaatgtaagcgtgacataactaattttacttcatttcaccgtgattg

SEQ ID NO: 38 - DNA, 73 bp, Artificial Sequence
  Oligonucleotide
agaataaacacaaaaacaaaaagtttttttaattttaatcaaaaaatgagtaagcgtaaagtcgccattatcg

SEQ ID NO: 39 - DNA, 65 bp, Artificial Sequence
  Oligonucleotide
gagggcgtgaatgtaagcgtgacataactaattacatgactcgagtcatgccgcttctcctgcct

SEQ ID NO: 40 - DNA, 67 bp, Artificial Sequence
  Oligonucleotide
gaataaacacaaaaacaaaaagtttttttaattttaatcaaaaaaatgtcagttattggtcgcattc

SEQ ID NO: 41 - DNA, 65 bp, Artificial Sequence
  Oligonucleotide
gagggcgtgaatgtaagcgtgacataactaattacatgactcgagttagaacattaccttatgac

SEQ ID NO: 42 - DNA, 63 bp, Artificial Sequence
  Oligonucleotide
tcaaggaagtaattatctactttttacaacaaatataaaacaatgtccgagcttaatgaaaag

SEQ ID NO: 43 - DNA, 66 bp, Artificial Sequence
  Oligonucleotide
aatgcttataaaactttaactaataattagagattaaatcgcttacatagattgagtgaaggtacg

SEQ ID NO: 44 - DNA, 27 bp, Artificial Sequence
  Oligonucleotide
aattagttatgtcacgcttacattcac

SEQ ID NO: 45 - DNA, 65 bp, Artificial Sequence
  Oligonucleotide
ggttttttcagttttgttctttttgcaaacagcggccgcctaaggggcgaattgggtaccggccg

SEQ ID NO: 46 - DNA, 32 bp, Artificial Sequence
  Oligonucleotide
taagcgatttaatctctaattattagttaaag

SEQ ID NO: 47 - DNA, 68 bp, Artificial Sequence
  Oligonucleotide
ctatgcggtgtgaaataccgcacagatgcgtaaggagaaaatacctgttccttaatcaaggatacctc

SEQ ID NO: 48 - DNA, 37 bp, Artificial Sequence
  Oligonucleotide
ccttaggcggccgctgtttgcaaaaagaacaaaactg

SEQ ID NO: 49 - DNA, 37 bp, Artificial Sequence
  Oligonucleotide
cattgttttatatttgttgtaaaaagtagataattac

SEQ ID NO: 50 - DNA, 69 bp, Artificial Sequence
  Oligonucleotide
gaataaacacaaaaacaaaaagtttttttaattttaatcaaaaaaatgaaattgttaaaacgagaaggc

SEQ ID NO: 51 - DNA, 70 bp, Artificial Sequence
  Oligonucleotide
cggatgtggggggagggcgtgaatgtaagcgtgacataactaattttacattcctcctttttcatatgac

SEQ ID NO: 52 - DNA, 62 bp, Artificial Sequence
  Oligonucleotide
catcaaggaagtaattatctactttttacaacaaatataaaacaatggcgagagtcataagc

SEQ ID NO: 53 - DNA, 66 bp, Artificial Sequence
  Oligonucleotide
cataaaaatgcttataaaactttaactaataattagagattaaatcgcttaattcaatgccggctc

SEQ ID NO: 54 - DNA, 771 bp, Klebsiella pneumoniae
atgaaaaaagtcgcacttgttaccggcgccggccaggggattggtaaagctatcgcccttcgtctggtgaaggatggatttgccgtggccattgccgattataacgacaccaccgccaaagcggtcgcctccgaaatcaaccaggccggcggccgcgccatggcggtgaaagtggatgtctccgaccgcgatcaggtgtttgccgccgtcgaacaggcgcgcaaaacgctgggcggcttcgacgtcatcgtcaacaacgccggcgtggcgccgtccacgccgatcgagtccattaccccggagattgtcgataaagtctacaacatcaacgttaaaggggtgatctggggcattcaggcggcggtcgaggcctttaagaaagagggtcacggcgggaaaatcatcaacgcctgttcccaggccggccacgtcggcaacccggagctggcggtatatagctcgagtaaattcgccgtacgcggcttaacccagaccgccgctcgcgacctcgcgccgctgggcatcacagtcaacggctactgcccggggattgtcaaaacgccaatgtgggccgaaattgaccgccaggtgtccgaagccgccggtaaaccgctgggttacggtaccgccgagttcgccaaacgcatcaccctcggccgcctgtccgagccggaagatgtcgccgcctgcgtctcctatcttgccagcccggattctgattatatgaccggtcagtcattgctgatcgacggcgggatggtgtttaactaa

SEQ ID NO: 55 - DNA, 1680 bp, Klebsiella pneumoniae
atggataaacagtatccggttcgtcagtgggcacatggtgcagatctggttgttagccagctggaagcacagggtgttcgtcagatttttggtattccgggtgccaaaatcgataaagtttttgatagcctgctggatagcagcattcgtattattccggtgcgtcatgaagcaaatgcagcatttatggcagcagcagttggtcgtattaccggcaaagccggtgttgcactggttaccagcggtccgggttgtagcaatctgattaccggtatggcaaccgcaaatagcgaaggtgatccggttgttgccctgggtggtgcagttaaacgtgcagataaagcaaaacaggttcatcagagcatggataccgttgcaatgtttagtccggttaccaaatatgcagttgaagttaccgcaccggatgcactggccgaagttgttagcaatgcatttcgtgcagcagaacagggtcgtccgggtagcagctttgttagcctgccgcaggatgttgttgatggtccggttagcggtaaagttctgcctgcaagccgtgcaccgcagatgggtgcagctccggatgatgcaattgatcaggttgcaaaactgattgcacaggccaaaaacccgatttttctgctgggtctgatggcaagccagccggaaaatagcgcagcactgcgtcgtctgctggaagcgagccatattccggttacaagcacctatcaggcagccggtgccgttaatcaggataactttagccgttttgcaggtcgtgttggtctgtttaataaccaggcaggggatcgcctgctgcaactggccgatctggttatttgtattggttattctccggtggaatatgaaccggcaatgtggaatagcggtaatgcaaccctggtgcatattgatgttctgccagcctatgaagaacgtaattatacaccggatgttgaactggtgggtgatattgcaggcaccctgaataaactggcacagaatattgatcatcgtctggttctgagtccgcaggcagcagaaattctgcgtgatcgtcagcatcagcgtgaactgctggatcgtcgtggtgcacagctgaatcagtttgcactgcatccgctgcgtattgttcgtgcaatgcaggatattgttaatagtgatgttaccctgaccgttgatatgggcagctttcatatttggattgcccgttatctgtatagctttcgtgcacgtcaggttatgattagcaatggtcagcagacaatgggtgttgcgctgccgtgggcaattggtgcatggctggttaatccggaacgtaaagttgtgagcgttagcggtgatggtggttttctgcaaagcagcatggaactggaaaccgcagttcgtctgaaagcaaatgttctgcatctgatttgggtggataatggctataatatggtggccatccaagaagagaaaaaataccagcgtctgagcggtgttgaatttggtccgatggattttaaagcctatgccgaaagttttggtgcaaaaggttttgccgttgaaagcgcagaagcactggaaccgaccctgcgtgcagcaatggatgtggatggtcctgccgttgttgcaattccggttgattatcgtgataatccgctgctgatgggtcagctgcatctgtcacagattctgtaa

SEQ ID NO: 56 - DNA, 930 bp, Saccharomyces cerevisiae
atgctgcgtagcctgctgcaaagcggtcatcgtcgtgttgttgcaagcagctgtgccacaatggttcgttgtagcagcagcagtaccagcgcactggcatataaacaaatgcatcgtcatgcaacccgtccgcctctgccgaccctggataccccgagctggaatgcaaatagcgcagttagcagcattatctatgaaacaccggcaccgagccgtcagcctcgtaaacagcatgttctgaattgtctggttcagaatgaaccgggtgttctgagccgtgttagcggcaccctggcagcacgtggttttaacattgatagcctggttgtttgcaacaccgaagttaaagatctgagtcgtatgaccattgttctgcaaggtcaggatggtgttgttgaacaggcacgtcgtcagattgaagatctggttccggtttatgcagttctggattataccaacagcgaaatcattaaacgtgaactggttatggcacgtattagtctgctgggcaccgaatattttgaggatctgctgctgcatcatcataccagcaccaatgccggtgcagcagatagccaagaactggttgcagaaattcgtgaaaaacagtttcatccggcaaatctgcctgcaagcgaagttctgcgtctgaaacatgaacatctgaacgatattaccaacctgaccaataactttggtggtcgtgtggttgatattagcgaaaccagctgtattgttgaactgagcgcaaaaccgacccgtattagcgcatttctgaaactggttgaaccgtttggtgttctggaatgtgcacgtagcggtatgatggcactgcctcgtacaccgctgaaaaccagcaccgaagaggcagcagatgaagatgaaaaaatcagcgaaattgtggatatcagccagctgcctccgggttaa